SEQUENCE LISTING (7 sequences)

SEQ ID NO: 1 - DNA, 8475 bp, Artificial Sequence
  synthetic
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatgctagcggaggaaaaactgtttcatacagaaggcgtggaggaaaaactgtttcatacagaaggcgtggaggaaaaactgtttcatacagaaggcgtagatctagactctagagggtatataatggaagctcgacttccagcttggcattccggtactgttggtaaaaagcttggcaatccggtactgttggtaaaggatccgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaagcggagctactaacttcagcctgctgaagcaggctggagacgtggaggagaaccctggacctatgcctgccatgaagattgagtgccgcatcacgggaaccctgaacggagtggagtttgagctggtcggaggtggagaagggactcctgagcagggacgtatgaccaacaagatgaagtctaccaagggcgccttgaccttctccccctaccttctctctcatgtcatgggatacgggttctaccactttggtacctatcccagtgggtatgagaatcccttcctgcatgccatcaacaacggggggtacaccaacaccaggattgagaagtatgaggatggaggagttcttcatgttagctttagctacagatatgaagcaggcagggtgattggggatttcaaggttgtcgggacaggattccctgaggacagtgtgatcttcaccgacaagatcatccggtccaatgctaccgtggagcacttgcacccaatgggagacaacgttcttgtgggctccttcgcgagaaccttttccctgagggatggaggctactactcatttgtggttgacagccacatgcacttcaagagtgccatccacccatccatcctccagaacggggggcccatgtttgccttcaggagagttgaggaacttcactccaacactgaacttggcattgtagagtatcaacatgccttcaagactcccatagcatttgcttaagcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 2 - DNA, 8565 bp, Artificial Sequence
  synthetic
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatgctagcggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtagatctagactctagagggtatataatggaagctcgacttccagcttggcattccggtactgttggtaaaaagcttggcaatccggtactgttggtaaaggatccgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaagcggagctactaacttcagcctgctgaagcaggctggagacgtggaggagaaccctggacctatgcctgccatgaagattgagtgccgcatcacgggaaccctgaacggagtggagtttgagctggtcggaggtggagaagggactcctgagcagggacgtatgaccaacaagatgaagtctaccaagggcgccttgaccttctccccctaccttctctctcatgtcatgggatacgggttctaccactttggtacctatcccagtgggtatgagaatcccttcctgcatgccatcaacaacggggggtacaccaacaccaggattgagaagtatgaggatggaggagttcttcatgttagctttagctacagatatgaagcaggcagggtgattggggatttcaaggttgtcgggacaggattccctgaggacagtgtgatcttcaccgacaagatcatccggtccaatgctaccgtggagcacttgcacccaatgggagacaacgttcttgtgggctccttcgcgagaaccttttccctgagggatggaggctactactcatttgtggttgacagccacatgcacttcaagagtgccatccacccatccatcctccagaacggggggcccatgtttgccttcaggagagttgaggaacttcactccaacactgaacttggcattgtagagtatcaacatgccttcaagactcccatagcatttgcttaagcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 3 - DNA, 8655 bp, Artificial Sequence
  synthetic
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatgctagcggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtagatctagactctagagggtatataatggaagctcgacttccagcttggcattccggtactgttggtaaaaagcttggcaatccggtactgttggtaaaggatccgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaagcggagctactaacttcagcctgctgaagcaggctggagacgtggaggagaaccctggacctatgcctgccatgaagattgagtgccgcatcacgggaaccctgaacggagtggagtttgagctggtcggaggtggagaagggactcctgagcagggacgtatgaccaacaagatgaagtctaccaagggcgccttgaccttctccccctaccttctctctcatgtcatgggatacgggttctaccactttggtacctatcccagtgggtatgagaatcccttcctgcatgccatcaacaacggggggtacaccaacaccaggattgagaagtatgaggatggaggagttcttcatgttagctttagctacagatatgaagcaggcagggtgattggggatttcaaggttgtcgggacaggattccctgaggacagtgtgatcttcaccgacaagatcatccggtccaatgctaccgtggagcacttgcacccaatgggagacaacgttcttgtgggctccttcgcgagaaccttttccctgagggatggaggctactactcatttgtggttgacagccacatgcacttcaagagtgccatccacccatccatcctccagaacggggggcccatgtttgccttcaggagagttgaggaacttcactccaacactgaacttggcattgtagagtatcaacatgccttcaagactcccatagcatttgcttaagcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 4 - DNA, 8660 bp, Artificial Sequence
  synthetic
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagctagcgaattcgaatttaaatcggatccgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaagcggagctactaacttcagcctgctgaagcaggctggagacgtggaggagaaccctggacctatgcctgccatgaagattgagtgccgcatcacgggaaccctgaacggagtggagtttgagctggtcggaggtggagaagggactcctgagcagggacgtatgaccaacaagatgaagtctaccaagggcgccttgaccttctccccctaccttctctctcatgtcatgggatacgggttctaccactttggtacctatcccagtgggtatgagaatcccttcctgcatgccatcaacaacggggggtacaccaacaccaggattgagaagtatgaggatggaggagttcttcatgttagctttagctacagatatgaagcaggcagggtgattggggatttcaaggttgtcgggacaggattccctgaggacagtgtgatcttcaccgacaagatcatccggtccaatgctaccgtggagcacttgcacccaatgggagacaacgttcttgtgggctccttcgcgagaaccttttccctgagggatggaggctactactcatttgtggttgacagccacatgcacttcaagagtgccatccacccatccatcctccagaacggggggcccatgtttgccttcaggagagttgaggaacttcactccaacactgaacttggcattgtagagtatcaacatgccttcaagactcccatagcatttgcttaagcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 5 - DNA, 9744 bp, Artificial Sequence
  synthetic
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgattctcgagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgctcctaggggatccttagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatggtggcggccgctaagcttgggctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaagctagcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactctagagctagcgaattcggtctcgccaccatggactttcaagtgcagatcttcagctttctgctgatctctgctagtgtcatcatgtctgaagtgcaactggtggaatctggaggtggagtggtgcaaccaggtaggtcactcaggctgtcttgctcagcctctggcttcacattctcaggatatggtctgagctgggtcagacaagcaccaggcaagggactggaatgggtcgctatgatcagcagtggaggatcttacacctactatgcagattctgtcaagggtaggtttgcaatctccagagacaatgccaagaacaccctgttcctgcagatggacagcctcagacctgaagatactggagtttacttctgtgctaggcacggagacgatccagcctggtttgcctactggggccaaggaactccagtgacagtctcttctgccagcactaagggtcctagcgtgtttccactggctcccagctccaagtcaacaagtggaggtacagcagctctcggatgtctggtgaaagattacttccctgagcctgtcacagtcagttctggaggtggaggcagtggaggtggtggatctggaggaggagggtctgacatccaactgacacagtcaccatcttcactgtctgccagcgtgggagacagggtcaccattacatgctctgtttcctcttccatatcatcaaacaatctccactggtatcaacagaagcctggtaaggctcccaaaccctggatctatggcacaagcaacctcgcatctggtgtgccatcacgcttctcaggctctgggagtggcacagactacacctttactatctcaagcctgcaacctgaggacattgctacctactattgccagcagtggtcttcatacccttacatgtacacctttggtcagggaactaaggtggaaatcaagagggaaatcgagcagaagctgatttccgaagaggatctcaatggagtgactgtcagctctgcactgtctaactccatcatgtacttctcacacttcgtgccagtgttcctccctgctaaacccaccacaactccagcacctagacctcccactccagcaccaactattgcatcccagcctctctccctcagaccagaagcatgcagacctgcagcaggtggagctgtgcacacaagaggtctggaccctttctgggtcctcgtggtggtgggtggagtcctggcatgttacagcctcctggtcaccgtggcattcatcatcttctgggtgagatctaagaggagcagactgctgcactctgattacatgaacatgacacccagaagacctggtcccaccagaaagcactaccaaccctacgcaccaccaagagactttgctgcatacagaagtctggagagggtcaagttctcaaggagtgcagatgctccagcctatcaacagggtcagaaccaactgtacaacgagctgaatctcggaagaagagaggagtacgatgtgctggataagagaagaggcagggacccagagatgggtgggaaacccagaagaaagaatcctcaagagggactgtacaatgagctgcagaaggataagatggctgaggcatactcagagatcggtatgaagggagagaggagaagaggcaaaggtcatgatggtctgtaccaaggtctgtccacagcaacaaaggatacatatgatgctctgcacatgcaggcactcccaccacggtgattaattaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 6 - DNA, 9759 bp, Artificial Sequence
  synthetic
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgattctcgagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgctcctaggggatccttagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatggtggcggccgctaagcttgggctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaagctagcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactctagagctagcgaattcggtctcgccaccatggactttcaagtgcagatcttcagctttctgctgatctctgctagtgtcatcatgtctgacatccaactgacacagtcaccatcttcactgtctgccagcgtgggagacagggtcaccattacatgctctgtttcctcttccatatcatcaaacaatctccactggtatcaacagaagcctggtaaggctcccaaaccctggatctatggcacaagcaacctcgcatctggtgtgccatcacgcttctcaggctctgggagtggcacagactacacctttactatctcaagcctgcaacctgaggacattgctacctactattgccagcagtggtcttcatacccttacatgtacacctttggtcagggaactaaggtggaaatcaagaggggaggaggtggcagtggaggaggaggctcaggtggaggtggatcaggtggtggtgggtccgaagtgcaactggtggaatctggaggtggagtggtgcaaccaggtaggtcactcaggctgtcttgctcagcctctggcttcacattctcaggatatggtctgagctgggtcagacaagcaccaggcaagggactggaatgggtcgctatgatcagcagtggaggatcttacacctactatgcagattctgtcaagggtaggtttgcaatctccagagacaatgccaagaacaccctgttcctgcagatggacagcctcagacctgaagatactggagtttacttctgtgctaggcacggagacgatccagcctggtttgcctactggggccaaggaactccagtgacagtctcttctgccagcactaagggtcctagcgtgtttccactggctcccagctccaagtcaacaagtggaggtacagcagctctcggatgtctggtgaaagattacttccctgagcctgtcacagtcagttctgaaatcgagcagaagctgatttccgaagaggatctcaatggagtgactgtcagctctgcactgtctaactccatcatgtacttctcacacttcgtgccagtgttcctccctgctaaacccaccacaactccagcacctagacctcccactccagcaccaactattgcatcccagcctctctccctcagaccagaagcatgcagacctgcagcaggtggagctgtgcacacaagaggtctggaccctttctgggtcctcgtggtggtgggtggagtcctggcatgttacagcctcctggtcaccgtggcattcatcatcttctgggtgagatctaagaggagcagactgctgcactctgattacatgaacatgacacccagaagacctggtcccaccagaaagcactaccaaccctacgcaccaccaagagactttgctgcatacagaagtctggagagggtcaagttctcaaggagtgcagatgctccagcctatcaacagggtcagaaccaactgtacaacgagctgaatctcggaagaagagaggagtacgatgtgctggataagagaagaggcagggacccagagatgggtgggaaacccagaagaaagaatcctcaagagggactgtacaatgagctgcagaaggataagatggctgaggcatactcagagatcggtatgaagggagagaggagaagaggcaaaggtcatgatggtctgtaccaaggtctgtccacagcaacaaaggatacatatgatgctctgcacatgcaggcactcccaccacggtgattaattaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 7 - DNA, 10251 bp, Artificial Sequence
  synthetic
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgattctcgagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgctcctaggggatccttagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatggtggcggccgctaagcttgggctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaagctagcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactctagagctagcgaattcggtctcgccaccatggagtttgggctgagctggctttttcttgtggctattttaaaaggtgtccagtgctcttccgacatccagctgacccagagcccaagcagcctgagcgccagcgtgggtgacagagtgaccatcacctgtagtgtcagctcaagtataagttccaacaacttgcactggtaccagcagaagccaggtaaggctccaaagccatggatctacggcacatccaacctggcttctggtgtgccaagcagattcagcggtagcggtagcggtaccgactacaccttcaccatcagcagcctccagccagaggacatcgccacctactactgccaacagtggagtagttacccgtacatgtacacgttcggccaagggaccaaggtggaaatcaaacgaggtggtggtggttctggtggtggtggttctggcggcggcggctccggtggtggtggatccgaggtgcagctggtggagagcggtggaggtgttgtgcaacctggccggtccctgcgcctgtcctgctccgcatctggcttcaccttcagcggctatgggttgtcttgggtgagacaggcacctggaaaaggtcttgagtgggttgcaatgattagtagtggtggtagttatacctactatgcagacagtgtgaagggtagatttgcaatatcgcgagacaacgccaagaacacattgttcctgcaaatggacagcctgagacccgaagacaccggggtctatttttgtgcaagacatggggacgatcccgcctggttcgcttattggggccaagggaccccggtcaccgtctcctcagcctccaccaagggcccatcggtcttccccctggcaccctcctccaagagcacctctgggggcacagcggccctgggctgcctggtcaaggactacttccccgaaccggtgacggtgtcgtcgtacgtcaccgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatgattaattaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

(19) United States
(12) Patent Application Publication
(10) Pub. No.: US 2023/0398215 A1
(43) Pub. Date: Dec. 14, 2023

(54) GENETICALLY ENGINEERED ANTIGEN-SPECIFIC NATURAL KILLER CELLS FOR IN SITU SYNTHESIS OF PROTEINS

(71) Applicant: SRI International, Menlo Park, CA (US)

(72) Inventor: Parijat Bhatnagar, Menlo Park, CA (US)

(73) Assignee: SRI International, Menlo Park, CA (US)

(21) Appl. No.: 18/033,907

(22) PCT Filed: Oct. 28, 2021
(86) PCT No.: PCT/US2021/057026

§ 371 Date: Apr. 26, 2023

Related U.S. Application Data
(60) Provisional application No. 63/106,838, filed on Oct. 28, 2020.

(51) Int. Cl. A61K 39/00

(52) U.S. Cl. CPC A61K 39/4631; A61K 39/4613; A61K 39/4622; A61K 39/464411; A61K 39/001112; A61K 39/4621; A61K 2239/21; A61K 2239/22

(57) ABSTRACT

An example genetically engineered natural killer (NK) cell comprises an exogenous polynucleotide sequence that includes a receptor element, an actuator element, and an effector element. The receptor element encodes a chimeric antigen receptor (CAR) comprising an extracellular antigen binding domain operably linked to a transmembrane domain, and an intracellular signaling domain, wherein the extracellular antigen binding domain recognizes a surface antigen of a target cell. The actuator element encodes a transcription factor binding site that upregulates synthesis of an effector protein. The effector element encodes the effector protein operably linked to a signal peptide, wherein, in response to the antigen binding domain of the CAR binding to the antigen of the target cell, the engineered NK cell is configured to activate and, to synthesize and secrete the effector protein.

### GOVERNMENT RIGHTS

This invention was made with Government support under grants 1DP2EB024245 and R21CA236640 awarded by the National Institutes of Health (NIH), and with Government support under contract number D19AP00024 awarded by the Defense Advanced Research Projects Agency (DARPA). The Government has certain rights in this invention.

### INCORPORATION-BY-REFERENCE OF MATERIAL SUBMITTED ELECTRONICALLY

Incorporated by reference in its entirety is a computer-readable nucleotide sequence listing, an ASCII text file which is 83 kb in size, submitted concurrently herewith, and identified as follows: “S1647132111_SequenceListing_ST25” and created on Oct. 28, 2021.

### BACKGROUND

Various standard-of-care therapeutics are designed to treat a disease at the time of diagnosis. Although many pathogens and diseased cells undergo dynamic changes in vivo, current drugs are not designed to co-evolve along with the in vivo disease microenvironment. Such therapeutics can include drugs administered in doses that are normalized to the body weight of the patient. However, disease burden can be different for similar-sized patients. If drug dosages are administered in excess, the therapeutic agents can end up in system circulation which can cause morbidity in normal tissue. In the case of suboptimal delivery, drug resistance may develop. While the patient can be monitored and the dosage adjusted based on health results, daily monitoring is costly. Additionally, monitoring strategies and treatments do not exist for many diseases. Thus, static therapeutics often cannot control dynamic pathogens and diseases that evolve and/or persist. The misalignment between the dynamic disease states and static therapeutics imposes a major social and economic burden. Several synthetic or host-defense peptides from other species have been identified for therapeutic effect in various diseases. However, such synthetic or host-defense peptides cannot be currently administered because they are rapidly degraded by the immune system and cause toxicity to normal (host) cells.

### SUMMARY

The present invention is directed to overcoming the above-mentioned challenges and others related to therapeutics for treating diseases, such as involving a genetically engineered natural killer (NK) cell line (such as NK-92MI) which can activate in situ to cause synthesis of a human or non-human therapeutic protein (effector) against the target disease.

Various embodiments of the present disclosure are directed to a genetically engineered NK cell comprising an exogenous polynucleotide sequence that includes, in operative association: a receptor element that encodes a chimeric antigen receptor (CAR) comprising an extracellular antigen binding domain operably linked to a transmembrane domain, and an intracellular signaling domain, wherein the extracellular antigen binding domain recognizes a surface antigen on a surface of a target cell; an actuator element that encodes a transcription factor binding site that upregulates synthesis of an effector protein in response to the antigen binding domain of the CAR binding to the antigen of the target cell; and an effector element that encodes the effector protein operably linked to a signal peptide. Wherein, in response to the antigen binding domain of the CAR binding to the antigen of the target cell, the engineered NK cell is configured to activate and, to synthesize and secrete the effector protein.

In some embodiments, the genetically engineered NK cell is configured to synthesize and secrete an amount of the effector protein as a function of an amount of the target cell present. In some embodiments, the amount of the effector protein is proportional to the amount of target cell present in situ.

In some embodiments, the signal peptide is upstream of the effector protein and is non-native to the effector protein. In some embodiments, the signal peptide is native to the effector protein.

In some embodiments, the intracellular signaling domain, the actuator element, and the signal peptide are constant domains and the extracellular antigen binding domain and the effector protein are variable domains.

In some embodiments, the actuator element is bound to the effector element and the NK cell includes a NK-92MI cell.

In some embodiments, the exogenous polynucleotide sequence includes the actuator element bound to the effector element bound to the receptor element.

In some embodiments, the effector protein is selected from a detectable reporter protein, a therapeutic protein, a downstream signaling protein, and a combination thereof.

In some embodiments, the intracellular signaling domain includes one or more of an intracellular signaling portion of a CD28, an intracellular signaling portion of a 4-1BB and an intracellular signaling portion of a CD3 zeta.

In some embodiments, the transcription factor binding site is selected from the group consisting of: a nuclear factor of activated T-cell (NFAT) response element, a serum response element (SRE), and a cyclic AMP response element (CRE).

Various embodiments are directed to a population of genetically engineered NK cells, each of the genetically engineered NK cells of the population comprising an exogenous polynucleotide sequence that includes an actuator element bound to an effector element bound to a receptor element, wherein the receptor element encodes a CAR comprising an extracellular antigen binding domain operably linked to a transmembrane domain, and an intracellular signaling domain, wherein the extracellular antigen binding domain recognizes a surface antigen on a surface of a target cell; the actuator element encodes a transcription factor binding site that upregulates synthesis of an effector protein in response to the antigen binding domain of the CAR binding to the antigen of the target cell; and the effector element encodes the effector protein operably linked to a signal peptide. Wherein, in response to the antigen binding domain of the CAR binding to the antigen of the target cell, the population of engineered NK cells are configured to activate and, in response, to synthesize and secrete a calibrated amount of the effector protein based on a presence of the target cell.

In some embodiments, the exogenous polynucleotide sequence includes the actuator element bound to and upstream from the effector element, and the effector element bound to and upstream from the receptor element, and wherein the signal peptide is upstream from the effector protein.

In some embodiments, the effector protein is a therapeutic protein that acts directly upon the target cell, the therapeutic protein being selected from the group consisting of: a cytotoxic protein, an immunostimulatory protein, and an immuno suppressive protein.

In some embodiments, the calibrated amount of the effector protein is a function of an amount of the target cell present in a plurality of cells or in a sample.

Various embodiments are directed to a method comprising contacting a plurality of cells with a volume of a genetically engineered NK cell, wherein the genetically engineered NK cell comprises a polynucleotide sequence that includes a receptor element that encodes a CAR comprising an extracellular antigen binding domain operably linked to a transmembrane domain, and an intracellular signaling domain, wherein the extracellular antigen binding domain recognizes a surface antigen on a surface of a target cell from the plurality of cells; an actuator element that encodes a transcription factor binding site; and an effector element that encodes an effector protein operably linked to a signal peptide. The method further includes, in response to contacting the plurality of cells with the genetically engineered NK cell and a presence of the target cell within the plurality of cells, causing binding of the receptor element to an antigen on a surface of the target cell; and in response to the antigen binding domain of the CAR binding to the antigen of the target cell, initiating expression of the effector element by the actuator element to synthesize the effector protein and the secretor peptide; and secreting the effector protein by the secretor peptide.

In some embodiments, the method further includes detecting expression of the effector protein, wherein detectable expression of the effector protein indicates the presence of the target cell.

In some embodiments, the method further includes, in response to the antigen binding domain of the CAR binding to the antigen of the target cell, activating the NK cell and, in response, synthesizing and secreting a calibrated amount of the effector protein based on the presence of the target cell

In some embodiments, the amount of the effector protein is proportional to an amount of the target cell present within the plurality of cells.

In some embodiments, the effector protein includes a therapeutic protein that acts directly on the target cell, and the method further includes neutralizing the target cell by the therapeutic protein.

### BRIEF DESCRIPTION OF THE DRAWINGS

Various example embodiments can be more completely understood in consideration of the following detailed description in connection with the accompanying drawings, in which:

FIG. 1 illustrates an example of a genetically engineered NK cell, in accordance with the present disclosure.

FIG. 2 illustrates an example of a genetically engineered NK cell and a sequence of events triggered when in a diseased environment, in accordance with the present disclosure.

FIG. 3 illustrates an example of a population of genetically engineered NK cells in a diseased environment, in accordance with the present disclosure.

FIG. 4 illustrates an example method of contacting a plurality of cells with a volume of a genetically engineered NK cell, in accordance with the present disclosure.

FIGS. 5A-5D illustrate example characterizations of genetically engineered NK cell function with specificity against Folate-receptor alpha (FRα) and mesothelin (MSLN) antigens, in accordance with the present disclosure.

FIGS. 6A-6H illustrate example cytolytic function of MSLN-specific and FRα-specific genetically engineered NK cells against target and non-target cells, in accordance with the present disclosure.

FIG. 7A-7D illustrate the artificial cell-signaling pathway of example genetically engineered NK cells, in accordance with the present disclosure.

FIGS. 8A-8B illustrate genetically engineered NK cells being redirected toward different cancer antigens, in accordance with the present disclosure.

FIGS. 9A-9F illustrate cytolytic function of example genetically engineered NK cells, in accordance with the present disclosure.

### DETAILED DESCRIPTION

In the following detailed description, reference is made to the accompanying drawings which form a part hereof, and in which is shown by way of illustration specific examples in which the disclosure can be practiced. It is to be understood that other examples can be utilized, and various changes may be made without departing from the scope of the disclosure. The following detailed description, therefore, is not to be taken in a limiting sense, and the scope of the disclosure is defined by the appended claims. It is to be understood that features of the various examples described herein may be combined, in part or whole, with each other, unless specifically noted otherwise.

In some embodiments, a cell can be engineered to express genetic elements including transmembrane receptor(s) that autonomously regulate the intracellular transcriptional machinery. Further, the genetic elements of the cell can be modular and/or a cell can include multiple genetic elements to yield an engineered cell having the capacity to serve as a vector for a variety of in vitro, ex vivo, and in vivo applications. Such cells can be modular in that parts can be conserved, and parts can be changed for different applications. The genetically engineered cells can be used for therapeutics and treatment methods that self-regulate the therapeutic response upon stimulation by the disease cells and that are applicable to a variety of cell-based diseases, including cancers, emerging pathogens, and others that evade the immune system or involve its malfunction. Multiple types of such genetically engineered cells, such as genetically engineered T-cells, provide a robust, reproducible cellular system to therapeutically target complex diseases in vivo. Such genetically engineered effector cells also provide a reliable in vivo imaging technology and a reliable, in vitro sensor technology in a variety of applications.

Despite recent approvals by various government agencies, such as the United States Food and Drug Administration (FDA), for cell therapies, the full impact of T-cell based drugs has been limited. This may be due to the autologous nature of adoptive cell therapies, which can contribute to manufacturing cost, product variability, and potential for adverse events. Furthermore, the dynamic state of cell-based pathologies and inter-patient variability present challenges for optimal dosing and can require continuous monitoring of the disease state for the individual patient. As noted above, drug delivery is often administered in doses normalized to body weight and surface area. However, disease burden can be different for similar sized patients. Embodiments in accordance with the present disclosure are directed to a genetically engineered NK cells which is used as a cellular chassis or vector to act as a biofactory for different target proteins. The engineered NK cell can be used to synthesize calibrated amounts of the target protein, and to induce autocrine and paracrine signaling due to artificial cell signaling. NK cells can exert a therapeutic effect quickly, such as exerting an anti-tumor effect and double time of around twenty-four hours, which can be useful for therapeutics.

Embodiments in accordance with the present disclosure include primary NK cells and NK cell lines that are genetically engineered with chimeric antigen receptors (CARs) to specifically detect (e.g., bind) antigens expressed on the surface of the target cell. By binding to the antigen, the genetically engineered NK cells may have improved functionality from natural NK cells. For example, tumor cells can develop resistance to NK cells by reduced expression of NK-cell activating ligands on the tumor cell surface. Further, the genetically engineered NK cells can have a comparatively more-effective design than CAR T-cell therapies. Somewhat surprisingly due to the differences between T-cells and NK cells and difficulty in manipulating primary NK cells, the genetically engineered NK cells can utilize at least some similar modular architecture to modified T-cells. As compared to T-cells, the genetically engineered NK cells can draw from the NK cell activation domains and improve in the artificial cell-signaling pathway. In various experimental embodiments involving studies of allogenic NK cells, and which was further surprising, the genetically engineered NK cells did not evidence graft verses host disease (GvHD), which illustrates that the NK cells can be used as a living vector for producing different target proteins. While NK cells have a relatively short half-life of around 1-2 weeks in vivo as compared to T-cells, the short half-life can provide the benefit of minimizing targeting of healthy host cells. As noted above, genetic manipulation and expansion of primary NK cells is difficult, particularly as compared to primary T cells. In many disease states, hosts (e.g., patients or other subjects) can have low T-cell numbers, as compared to NK cells and NK cells may have reduced adverse events due to cytokine release syndrome, neurotoxicity, and GvHD.

In various embodiments, the genetically engineered NK cell is modular and antigen-specific. Antigen-specificity can be used to overcome tumor resistance and directs the cytolytic function toward different antigen-presenting target cells, such as host cells of a human or other organism. Further, the artificial cell-signaling pathway of such genetically engineered NK cells can introduce the capability to serve as vector by producing calibrated amounts of protein-based therapeutics and inducting intended autocrine and paracrine signaling, upon the genetically engineered NK cell engaging the target antigen. The genetically engineered NK cell can allow for focused synthesis of the biologics at the target site and/or extend treatment duration for better patient outcome by limiting systemic toxicity.

Various embodiments demonstrate the successful implementation of the artificial cell-signaling pathway in an NK cell line. An example NK cell line includes NK-92MI. NK-92MI is a fast growing cytolytic cell line with a track record of clinical efficiencies. In some experimental embodiments, the NK-92MI cell line was transformed into a vector for engaging antigen-presenting target cells and to trigger the synthesis of calibrated amounts of engineered proteins in situ, herein sometimes referred to as “effector proteins”. The genetically engineered NK cell can provide an allogenic living vector that is modular. For example, the modularity can be used to combine different receptor elements with different effector elements, and which allows for reprogramming the NK cells to target diseases with known biomarkers, such as cancer, viral infections, and/or autoimmune disorders.

As used herein, a “genetically engineered NK cell” includes and/or refers to an NK cell that is genetically engineered or modified to comprise a (i) receptor element, (ii) actuator element, and (iii) effector element, each of which can be modular. As used herein, the terms “modular” and “modularity” include and/or refer to the versatility associated with recombinant sequence domains and the resulting recombinant polypeptides when assembled in various combinations for introduction into an engineered NK cell. As used herein, “receptor element” includes and/or refers to a polynucleotide sequence encoding a transmembrane receptor, such as a CAR, capable of a specific interaction with a target cell. Depending on the particular application, the receptor element can be reprogrammed by exchanging the single chain variable fragment (scFV) portion of CAR for an extracellular antigen binding domain specific for a different disease-associated antigen. Other receptor elements that can be used include, without limitation, CARs having specificity for antigens associated with autoimmune disorders, CARs having specificity for antigens associated with neural disorders (e.g., PTSD, Parkinson's disease, Alzheimer's disease), ligand-gated GPCRs (e.g., GPR1 Glucose receptor), light-gated ion channels (e.g., melanopsins, rhodopsins, photopsins), pressure sensing ion channels (e.g., TRPV1, TRPV2), and ligand-gated ion channels.

As used herein, “actuator element” includes and/or refers to a polynucleotide sequence encoding a transcription factor binding site that initiates transcription and translation events downstream of a triggering signal (e.g., binding of the sensing element to a target antigen). In general, the underlying molecular mechanism of the actuator element is based on the intracellular calcium [Ca²⁺]ᵢ dynamics, a mechanism used by almost all types of cells to regulate their functions. Exemplary response elements include, without limitation, NFAT (“nuclear factor of activated T cells”) response element (NFAT-RE), serum response element (SRE), and cyclic AMP response element (CRE).

As used herein, “effector element” includes and/or refers to a polynucleotide sequence encoding an effector protein, and in some instances, an effector protein operably linked to a signal peptide The polynucleotide sequence encoding the effector protein can be, for example, a sequence derived from a human gene, a sequence derived from a gene of a non-human species, a recombinant sequence, a sequence encoding a detectable reporter molecule, a sequence encoding a detectable imaging molecule, a sequence encoding a therapeutic molecule, and the like.

The genetically engineered NK cell into which the receptor element, the actuator element, and the effector element are introduced can be any NK cell type including human NK cells or non-human NK cells (e.g., mammal, reptiles, plants, among others). In this manner, the genetically modified cellular “source” of the modular elements provides a cellular chassis or frame providing, among other things, transcriptional and translational machinery for expression and presentation of the receptor element, the actuator element, and the effector element. In some embodiments, the NK cells can be from a source (e.g., a first human), modified, and administered to an organism that is different than the source (e.g., the host which is a second human) In other embodiments, the NK cells can be from the source (e.g., a first human), modified, and administered back to the source (e.g., the source is the host).

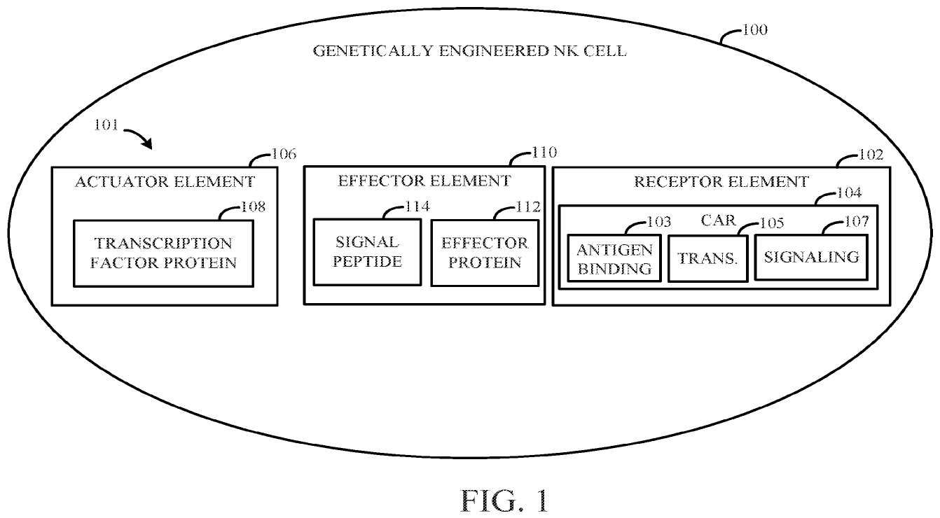

Turning now to the figures, FIG. 1 illustrates an example genetically engineered NK cell, in accordance with the present disclosure. The genetically engineered NK cell 100 can be modular in that elements can be adjusted for different target cells and to synthesize different effector proteins.

The genetically engineered NK cell 100 comprises an exogenous polynucleotide sequence that includes, in operative association, a receptor element 102, an actuator element 106, and an effector element 110. A variety of different types of NK cells can be used, such as an NK cell from the NK-92MI cell line, sometimes referred to as a NK-92MI cell.

The receptor element 102 encodes a CAR 104. A CAR is sometimes called a “chimeric receptor”, a “T-body”, or a “chimeric immune receptor (CAR).” As used herein, a CAR includes and/or refers to an artificially constructed hybrid protein or polypeptide comprising extracellular antigen binding domain(s) 103 of an antibody (e.g., scFv) operably linked to a transmembrane domain 105 and at least one intracellular signally domain 107. For example, the CAR 104 includes an extracellular antigen binding domain 103 operably linked to the transmembrane domain 105, and the intracellular signaling domain 107. The CAR 104 can be designed to identify a surface antigen of a target, such as a target cell of a host. The CAR 104 can mobilize internal Ca⁺² stores for intracellular Ca⁺² release in response to antigen binding. For example, the extracellular antigen binding domain 103 of the CAR 104 can recognize a surface antigen on a surface of a target cell, such as diseased cells of a host.

As used herein, the extracellular antigen binding domain 103 includes and/or refers to a polynucleotide sequence that is complementary to the surface antigen of the target cell. The extracellular antigen binding domain 103 can bind to the surface antigen of a target cell, as described above.

The transmembrane domain 105 includes and/or refers to a polynucleotide sequence encoding a transmembrane segment of a transmembrane protein, e.g., a type of membrane protein that spans the membrane of a cell, such as the membrane of the NK cell 100. The transmembrane domain 105 can be derived from a natural polypeptide, or can be artificially designed. A transmembrane domain 105 derived from a natural polypeptide can be obtained from any membrane-binding or transmembrane protein. For example, a transmembrane domain of a T cell receptor a or 13 chain, a CD3 chain, CD28, CD3ε, CD45, CD4, CD5, CD8, CD9, CD16, CD22, CD33, CD37, CD64, CD80, CD86, CD134, CD137, ICOS, CD154, or a GITR can be used.

The intracellular signaling domain 107 includes and/or refers to a polynucleotide sequence encoding any oligopeptide or polypeptide known to function as a domain that transmits a signal to cause activation or inhibition of a biological process in a cell. Example intracellular signaling domains include an intracellular signaling portion of a CD28, an intercellular signaling portion of a 4-1BB, and an intracellular signal portion of a CD3-zeta. In some embodiments, the intracellular signaling domain 107 includes the intracellular signaling portion of CD28, the intercellular signaling portion of 4-1BB, and the intracellular signal portion of CD3-zeta. However, embodiments are not so limited and can include other types and combinations of intracellular signaling domains. For example, the intracellular signaling domain 107 can include encode any molecule that can transmit a signal into a cell when the extracellular antigen binding domain 103 present within the same molecule binds to (interacts with) an antigen.

Generally, the antigen binding domain 103 of a CAR 104 has specificity for a particular antigen expressed on the surface of a target cell of interest. As described above, the extracellular binding domain 103 capable of binding to an antigen includes any oligopeptide or polypeptide that can bind to the antigen, and includes, for example, an antigen-binding domain of an antibody and a ligand-binding domain of a receptor. The extracellular antigen binding domain 103 binds to and interacts with the antigen, for example, an antigen present on a cell surface, and thereby imparts specificity to an genetically engineered NK cell 100 expressing the CAR 104. In some embodiments, the receptor element 102 encodes a CAR 104 comprising an extracellular antigen binding domain 103 having specificity for Folate-Receptor alpha (FRa), which is an antigen found to be overexpressed on various cancers including ovarian, cervical, lung, breast, kidney, and brain. Other chimeric antigen receptors appropriate for use as the antigen binding portion of the receptor element 102 include those having specificity for a subset of immune cells, for one or more tumor antigens, and/or for one or more viral antigens.

The actuator element 106 encodes a transcription factor binding site 108. The transcription factor binding site 108 includes and/or refers to binding site for a protein that upregulates synthesis of an effector protein 112 in response to the extracellular antigen binding domain 103 of the CAR 104 binding to the antigen of the target cell. The transcription factor binding site 108 can bind to transcription factors as triggered by [Ca²⁺], which as described above, are caused to release in response to the antigen binding. In some embodiments, the transcription factor binding site 108 is selected from a nuclear factor of activated T-cell (NFAT) response element, a serum response element (SRE), and a cyclic AMP response element (CRE). The actuator element 106 can thereby include a sequence for binding the factors triggered by [Ca²⁺], and can trigger amplified synthesis of the effector protein 112 in response to [Ca2⁺]ᵢ rise.

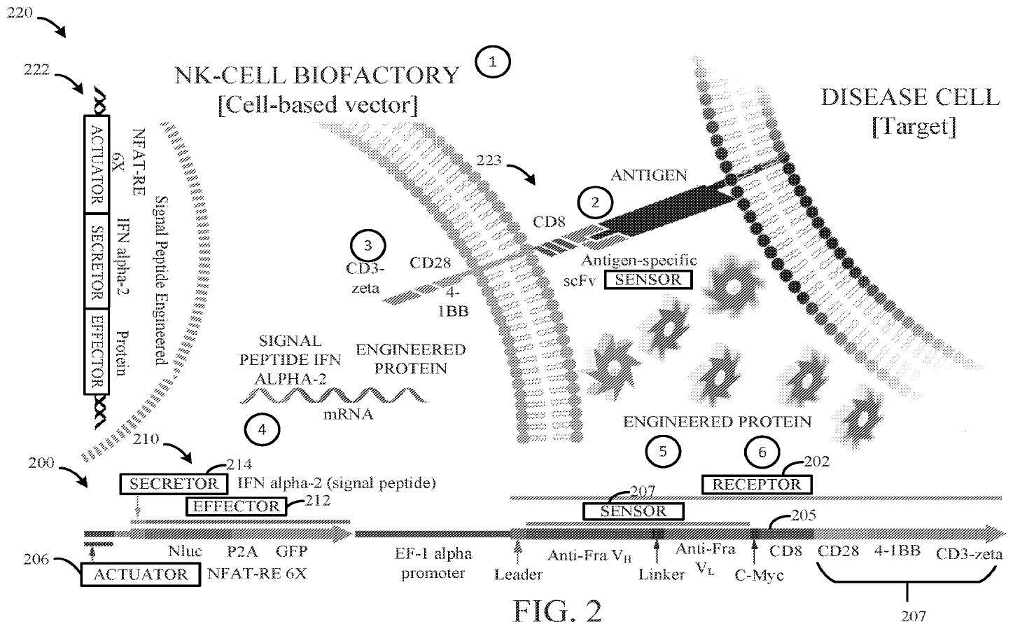

In some embodiments, the actuator element 106 encodes a NFAT transcription factor binding site for a transcription factor protein. NFAT transcription factor family consists of five members NFATc1, NFATc2, NFATc3, NFATc4, and NFAT5. For review, see Sharma S et al. (2011) PNAS, 108(28); Hogan P G et al. (2010) Ann Rev Immunol, 28; Rao A, Hogan P G (2009) Immunol Rev, 231(1); Rao A (2009) Nat Immunol, 10(1), M. R. Müller and A. Rao, Nature Reviews Immunology, 2010, 10, 645-656; M. Oh-Hora and A. Rao, Curr. Opin. Immunol., 2008, 20, 250-258. Crabtree & Olson E N (April 2002), Cell 109 Suppl (2): S67-79, which are each hereby incorporated herein in their entireties for their teaching. NFATc1 through NFATc4 are regulated by calcium signaling. Calcium signaling is critical to NFAT activation because calmodulin, a well-known calcium sensor protein, activates the serine/threonine phosphatase calcineurin. The underlying molecular mechanism of this strategy is based on intracellular Ca⁺² ([Ca²⁺]ᵢ) dynamics (as further shown by FIG. 2). The [Ca²⁺]ᵢ dynamics are common to almost all cell types, and the approach is thus broadly applicable. The [Ca²⁺]ᵢ rise from CAR-mediated stimulation of cells leads to dephosphorylation of the nuclear factor of an activated NK cell 100 proteins (through Ca⁺²/calmodulin-dependent serine phosphatase calcineurin), which then translocated to the nucleus and interact with the NFAT Response Element (NFAT-RE) to upregulate expression of the effector protein 112. In parallel, the NFAT-RE also performs its natural function of inducing IL-2 in the activated genetically engineered NK cell 100 that regulates clonal expansion proportional to the disease burden. The expression of a NFAT-RE induced reporter protein can also be used to quantitatively assess the level of activation of a genetically engineered NK cell 100.

The effector element 110 encodes the effector protein 112, and in some instances, encodes the effector protein 112 operably linked to a signal peptide 114. As further illustrated herein, in some embodiments, the signal peptide 114 is upstream of the effector protein 112. The signal peptide 114 can be non-native to the effector protein 112. For example, the effector protein 112 can be unable to secrete into the extracellular environment without the addition of the signal peptide 114. However, embodiments are not so limited and in some embodiments, the effector protein 112 includes a native signal peptide. For example, the effector protein 112 can (natively) include the signal peptide 114.

As used herein, the terms “secretor”, “secretory peptide and “signal peptide” are used interchangeable and include and/or refer to a peptide that assists or directs the synthesized effector protein 112 into the extracellular environment (e.g., assists with translocating the effector element 110). The signal peptide 114 can be operably linked or fused to the effector protein 112 for release into the extracellular environment. In this manner, the signal peptide 114 can direct movement of the effector protein 112 outside of the genetically engineered NK cell 100. A signal peptide 114 is particularly advantageous when included in the genetically engineered NK cell 100 expressing an effector protein 112 that is unable to and/or minimally able to translocate natively, where the effector protein 112 may remain inside the genetically engineered NK cell 100 in the absence of the signal peptide 114 and/or can translocate at a rate below a threshold. Generally, signal peptides are located at the N-terminus of nascent secreted proteins and characteristically have three domains: (1) a basic domain at the N-terminus, (2) a central hydrophobic core, and (3) a carboxy-terminal cleavage region. Any appropriate signal peptide can be used. For example, the signal peptide 114 can be the signal peptide of Interleukin-6 (IL-6) or Interleukin-2 (IL-2).

In various embodiments, in response to the extracellular antigen binding domain 103 of the CAR 104 binding to the antigen of the target cell (e.g., a target host cell), the engineered NK cell 100 is configured to activate, and to synthesize and secrete the effector protein 112. For example, the genetically engineered NK cell 100 can synthesize and secrete an amount of the effector protein 112 as a function of an amount of the target cell present in the environment (e.g., the extracellular environment), such as secreting an amount of the effector protein 112 in the environment that is proportional to the number of target cells present in the environment.

The effector protein 112 can include a variety of different types of proteins, which can be used to provide therapy to a host, such as a patient. For example, the effector protein 112 can include a detectable reporter protein, a therapeutic protein, a downstream signaling protein, and a combination thereof. As used herein, a detectable reporter protein includes and/or refers to a protein that is detectable upon expression, such as a protein that provides an optical, electrical or other type of detectable signal. A therapeutic protein includes and/or refers to a protein that provides a therapeutic effect to the patient. A downstream signaling protein includes and/or refers to a protein that drives downstream elements of a signaling pathway, such as for regulation of cell growth, proliferation, differentiation, and apoptosis.

Non-limiting examples of effector proteins include cytotoxic polypeptides of bacterial origin (e.g., parasporin, plantaricin A); insect origin (e.g. Polybia-MP1); antiviral polypeptides from viral origin (e.g., α-helical peptide (AHP)); antiviral polypeptides from viral origin (e.g., anti-viral peptides (AVP)); immunosuppressive peptides of fungal origin (e.g., colutellin A); vasodilators (e.g., relaxin, bradykinin) and endopeptidase (e.g., heparanase, relaxin, collagenase); and cell-penetrating cationic peptides (e.g., LL-37, TAT peptide). With respect to colutellin A, self-reactive NK cells can be engineered from NK cells obtained from hosts or other sources having autoimmune disorders (e.g., type 1 diabetes, polymyositis, and lupus). In particular, the NK cells can be engineered for localized expression of colutellin A upon stimulation by target self-antigens. Systemic infusion of immunosuppressive agents cannot be used in hosts with these conditions due to the risk of other opportunistic infections. With respect to vasodilators and endopeptidases, such NK cells can be used to improve perfusion (see Chauhan V P & Jain R K (2013) Nat. Mater. 12(11):958-962) and assist in efficient delivery of anticancer agents that cannot be systemically administered as they damage structural tissues and are tumorigenic. With respect to cell-penetrating cationic peptides, these target peptides can be used to target intracellular bacteria. For example, site-specific overexpression of such peptides can be a potent therapy for tuberculosis.

As noted above, in some embodiments, the effector protein 112 is a therapeutic protein. The therapeutic protein can act directly on the target cell, in some embodiments. In other embodiments, the therapeutic protein can act on cells adjacent to the target cell or on non-cellular components. Example therapeutic proteins include a cytotoxic protein, an immunostimulatory protein, and an immunosuppressive protein.

Different parts of the genetic elements 102, 106, 110 of the genetically engineered NK cell 100 can be modular and other parts can be conserved (e.g., may not change for different implementations). For example, in some embodiments, the intracellular signaling domain 107, the actuator element 106, and the signal peptide 114 are constant domains, and the extracellular antigen binding domain 103 and the effector protein 112 are variable domains. As an example, the extracellular antigen binding domain 103 can be changed for different targets and/or the effector protein 112 can be changed to cause in situ synthesis of different proteins, while the intracellular signaling domain 107, the actuator element 106, and the signal peptide 114 remain the same for the different implementations. Keeping parts conserved can reduce production time. However, embodiments are not so limited, and any part of the genetically engineered NK cell 100 can be modified.

In some embodiments, the genetically engineered NK cell 100 can include multiple (e.g., two or more) of some or all of the genetic elements 102, 106, 110. For example, the genetically engineered NK cell 100 can include multiple receptor elements 102, multiple actuator elements 106, and/or multiple effector elements 110. In some embodiments, multiplicity takes the form of providing multiple genetically engineered NK cells (e.g., a plurality of cells) modified as described herein to a host to provide more than one therapeutic task for treating or preventing a disease and/or for other purposes.

In some embodiments, the actuator element 106 is bound to the effector element 110. In some embodiments, the exogenous polynucleotide 101 includes the actuator element 106 bound to the effector element 110 bound to the receptor element 102. For example, the exogenous polynucleotide sequence 101 can include the actuator element 106 bound to and upstream from the effector element 110, and the effector element 110 bound to and upstream from the receptor element 102, wherein the signal peptide 114 is upstream from the effector protein 112.

FIG. 2 illustrates an example of a genetically engineered NK cell and a sequence of events triggered when in a diseased environment, in accordance with the present disclosure. The genetically engineered NK cell can be used as or act as a living vector to synthesize the effector protein 212 using the artificial cell-signaling pathway and/or to trigger a sequence of events 220. The genetically engineered NK cell 200 synthesizes the engineered effector protein 212 in situ upon interacting with the antigen-presenting target cell, as shown at 222.

As previously described, the genetically engineered NK cell 200 comprises the receptor element 202 encoding the extracellular antigen binding domain 203, transmembrane domain 205, and the intracellular signaling domain 207, the actuator element 206 encoding the transcription factor binding site (e.g., NFAT), and the effector element 210 encoding the effector protein 212 and, optionally, the signal peptide 214. The genetically engineered NK cell 200 can comprise a single plasmid (e.g., a single construct including each of) comprising three constant domains (e.g., the actuator element 206, the signal peptide 214, and portions of the receptor element 202, such as the transmembrane domain 205 and the intracellular signaling domain 207), and two variable domains (e.g., the antigen binding domain 203 (labeled as the “sensor”) and effector protein 212) arranged in cis.

The constant domains can be configured to provide functionality to the genetically engineered NK cell 200. The constant domains form part of the intracellular signaling pathway and include a transmembrane molecule (e.g., transmembrane domain 205) that mobilizes the calcium-dependent transcriptional machinery (e.g., actuator element 206) to upregulate the effector transgene (e.g., effector protein 212) fused to the signal peptide 214 that assists in transporting the effector transgene into the extracellular space 223.

The variable domains can be responsible for the applicability of the genetically engineered NK cell 200 to a variety of different diseases, target cells, therapy, and/or other applications. For example, the variable domains can impart specificity to the genetically engineered NK cell 200 against particular diseases. The variable domains can include a variable heavy-light (V_{H}-V_{L}) chain (e.g., the antigen binding domain 203, labeled as the “sensor” of the receptor element 202) to identify the antigen biomarker on the target cell (e.g., labeled “disease cell”) independent of the peptide-major histocompatibility complex, and the effector transgene (e.g., effector protein 212). The variable domains are modular. For example, the antigen binding domain 203 can be exchanged or revised to reprogram the genetically engineered NK cell 200 to target biomarkers specific to different cell-based diseases. As another example, the effector protein 212 can be exchanged or revised with different therapeutic transgenes, such as for neutralizing the pathology that activated the genetically engineered NK cell 200 and essentially creating an off-shelf living vector, which is enhanced further by the innate cytolytic activity of NK cells.

In some embodiments, the receptor element 202 encodes a CAR. Characteristics of CARs include their ability to redirect T-cell specificity and reactivity toward a selected target in a non-MHC-restricted manner, exploiting the antigen-binding properties of monoclonal antibodies. Somewhat surprisingly, CARs similarly provide such abilities to NK cells. The non-MHC-restricted antigen recognition gives NK cells expressing CARs the ability to recognize antigen independent of antigen processing, thus bypassing a major mechanism of tumor escape. Referring to FIG. 2, expression of a transmembrane CAR enables a genetically engineered NK cell 200 to sense and bind to the target antigen expressed on the surface of target cell. Binding of the CAR and target surface antigen on the target cell activates the genetically engineered NK cell 200, which triggers an activation cascade leading to the expression of the effector protein 212, such as an engineered reporter, imaging, and/or therapeutic protein. For example, expression of the effector protein 212 is autonomously expressed as part of the NK cell 200 activation cascade in response to binding of the transmembrane receptor to the antigen presented on the target cell.

More particularly, the genetically engineered NK cell 200 expressing a CAR can bind to a specific antigen via the CAR, and in response a signal is transmitted into the NK cell 200, and as a result, the NK cell 200 is activated. The activation of the NK cell 200 expressing the CAR is varied depending on the kind of target cell and an intracellular domain of the CAR, and can be confirmed based on, for example, release of a cytokine, improvement of a cell proliferation rate, change in a cell surface molecule, or the like as an index. For example, release of a cytotoxic cytokine (e.g., tumor necrosis factor, a lymphotoxin, etc.) from the activated NK cell 200 causes destruction of a target cell expressing an antigen. In addition, release of a cytokine or change in a cell surface molecule stimulates other immune cells, for example, a B cell, a dendritic cell, a NK cell, and a macrophage.

As shown by FIG. 2, an example sequence of events 220 triggered by or related to the genetically engineered NK cell 200 includes (1) the NK cell 200 actively migrating to the diseased environment, (2) the CAR on the NK cell 200 surface engaging the antigen of the target cell, (3) the NK cell activation, (4) upregulation of the effector protein 212 with the signal peptide 214 through the NFAT, (5) signal peptide 214 is cleaved off and effector protein 212 is transported into the extracellular space 223, and (6) antigen stimulation regulates cytokines that modulate cell expansion in response to the disease burden.

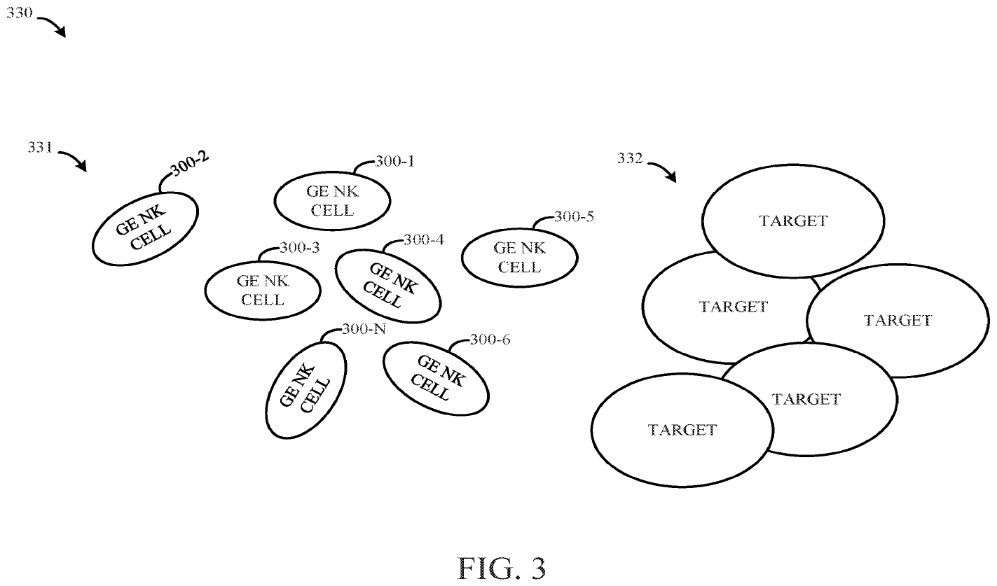

FIG. 3 illustrates an example of a population of genetically engineered NK cells in a diseased environment, in accordance with the present disclosure. The population 331 can include a plurality of genetically engineered NK cells 300-1, 300-2, 300-3, 300-4, 300-5, 300-6, 300-N (herein generally referend to as “the genetically engineered NK cells 300” for ease of references). Each of the genetically engineered NK cells 300 can include at least substantially the same features and elements as the genetically engineered NK cell 100 of FIG. 1, the details of which are not repeated.

In the example illustrated by FIG. 3, the environment is an extracellular space 330 that includes (a presence of) target cell(s) 332, such that the space 330 can be referred to as a diseased environment. The population 331 of the genetically engineered NK cells 300 can bind to the antigens of the target cell(s) 332 via the antigen binding domain of the CAR. In response to the binding, the genetically engineered NK cells 300 can activate and, in response, synthesize and secrete a calibrated amount of the effector protein based on a presence of the target cell(s) 332. For example, the calibrated amount of the effector protein is a function of an amount of the target cell 332 present in a plurality of (host) cells, such as in an extracellular space 330 or in a sample. As previously described, the calibrated amount of the effector protein can be proportional to the amount of the target cell 332. Although the extracellular space 330 illustrates genetically engineered NK cells 300 and the target cells 332, the extracellular space 330 and the plurality of (host) cells can further include other normal and/or diseased cells, among other non-cellular components.

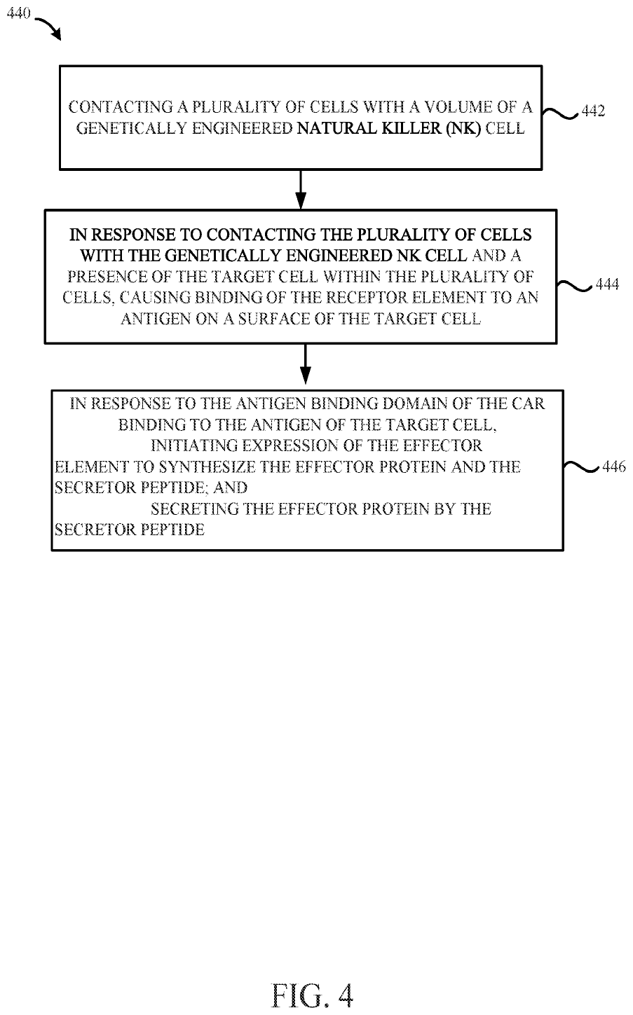

FIG. 4 illustrates an example method of contacting a plurality of cells with a volume of a genetically engineered NK cell, in accordance with the present disclosure. The method 440 can be implemented using the genetically engineered NK cell 100 illustrated by FIG. 1 and/or the population 331 of genetically engineered NK cells 300 illustrated by FIG. 3.

At 442, the method 440 includes contacting a plurality of cells with a volume of a genetically engineered NK cell. The cells can be contacted by contacting a sample with or administering the volume of the genetically engineered NK cell to a host, such as a patient. The genetically engineered NK cell can include at least some of substantially the same features and components as previously described by the genetically engineered NK cell 100 of FIG. 1, the details of which are not repeated.

At 444, in response to contacting the plurality of cells with the genetically engineered NK cell and a presence of the target cell within the plurality of cells, the method 440 includes causing binding of the receptor element to an antigen on a surface of the target cell. The plurality of cells, including the target cell, can include cells of a host (e.g., host cells and target host cells).

At 446, in response to the antigen binding domain (of the CAR) binding to the antigen of the target cell, the method 440 includes initiating expression of (e.g., transcription and translation of) the effector element by the actuator element to synthesize the effector protein and the signal peptide, and secreting the effector protein by the signal peptide. In some embodiments, the method 440 can further include, in response to the antigen binding domain of the CAR binding to the antigen of the target cell, activating the NK cell and, in response, synthesizing and secreting a calibrated amount of the effector protein based on the presence of the target cell. As previously described, the calibrated amount of the effector protein can be a function of (e.g., is proportional to) an amount of the target cell present within the plurality of cells in the environment.

In some embodiments, the method 440 further includes detecting expression of the effector protein. Detectable expression of the effector protein can indicate the presence of the target cell. In some embodiments, as described above, the effector protein includes a therapeutic protein. The therapeutic protein can act directly on the target cell, such as killing the target cell. For example, the method 440 can include neutralizing the target cell by the therapeutic protein. In other embodiments, the therapeutic protein can additionally and/or alternatively act on cells adjacent to the target cells or on the non-cellular components, such as providing a chemotactic gradient that other immune cells can follow for an inflammatory response and for infiltrating cold tumors.

Various embodiments are directed to a pharmaceutical composition comprising a genetically engineered NK cell and a pharmaceutically acceptable carrier or excipient, such as the genetically engineered NK cell 100 of FIG. 1 and/or the population 331 of genetically engineered NK cells 300 of FIG. 3.

For example, a NK cell composition, such as a pharmaceutical composition, can comprises a plurality of the genetically engineered NK cells described herein and an acceptable carrier, diluents, or excipient (e.g., a pharmaceutically acceptable carrier, diluent, excipient or a combination thereof). The means of making such a composition have been described in the art (see, for instance, Remington's Pharmaceutical Sciences, 16th Ed., Mack, ed. (1980)). Preferably, the composition is prepared to facilitate the administration of the NK cells into a living organism. In some embodiments, the pharmaceutical composition comprises a plurality of genetically engineered NK cells as described herein and, for example, a balanced salt solution, preferably Hanks' balanced salt solution, or normal saline.

Some embodiments are directed to methods of forming the genetically engineered NK cells, such as genetically engineering or modifying an NK cell to include the components and features as described by the genetically engineered NK cell 100 of FIG. 1.

The genetically engineered NK cells and cell compositions provided herein have properties advantageous for use in a variety of in vitro, ex vivo, and in vivo applications. For example, in vitro uses of the NK cells and cell compositions provided herein include, without limitation, detecting target cells on the basis of antigens expressed on the surface of the target cells. The target cell can be a cancer cell (e.g., tumor cell), a cell infected by a pathogen such as a virus or bacterium, a cell type associated with an autoimmune disorder (e.g., Type 1 diabetes, lupus), a cell type associated with a neurodegenerative disease such as Alzheimer's Disease, ALS, or Huntington's Disease. Also, the target (host) cell can be a cell type associated with any other pathology for which the affected (host) cell having aberrant expression of a cell surface antigen relative to an unaffected (host) cell. Methods for using the genetically engineered NK cells or cell compositions for in vitro target cell detection are described below.

Ex vivo uses of the genetically engineered NK cells and cell compositions provided herein include, without limitation, early disease detection and companion diagnostic or therapeutic applications for the disease target cells identified on the basis of antigens expressed on the surface of the disease target cells. For example, the NK cells can be used for ex vivo applications in companion diagnostics for cancer immunotherapy. By way of example, the NK cell engineered with NFAT_RE6X→Nluc-2A-GFP can be engineered to express different types of CARs. The expression of Nluc when CAR engages its target antigen versus the non-specific Nluc expression can inform on the comparative and quantitative robustness of each CAR for its efficiency to cause the intended on-target effect versus unintended off-target effects. Methods for using the genetically engineered NK cells or cell compositions in ex vivo therapeutic applications are described further below. Other ex vivo applications of the genetically engineered NK cells and cell compositions include, without limitation, applications for companion diagnostics for cell therapies for treating infectious diseases, autoimmune disorders, neurodegenerative disorders, and other cell-based pathologies associated with aberrant expression of a cell surface antigen relative to an unaffected (host) cell.

In vivo applications of the genetically engineered NK cells and cell compositions provided herein include, without limitation, in vivo imaging of disease sites, in vivo methods for localized therapy at a disease site (e.g., targeted therapy for ovarian cancer) or site of pathogen infection (e.g., targeted therapy for cells infected by dengue virus, Zika virus, West Nile virus, yellow fever, HIV, or a hepatitis virus (e.g., HepB, HepC)).

Various embodiments are directed to a panel of different types of genetically engineered NK cells, such as a plurality of NK cells engineered with different effector proteins and/or extracellular antigen binding domains (among other differences), and which are used to simultaneously target different cells and/or secrete different effector proteins.

In some embodiments, a method of detecting a target cell comprises (a) contacting a genetically engineered NK cell to a cell population, and (b) detecting expression of the effector protein, wherein detectable expression of the effector protein indicates the presence of the target cell of interest. In some embodiments, the NK cell includes a NFAT response element and a reporter protein, and in the presence of the target cell in the contacted cell population, the genetically engineered NK cell binds to a surface molecular antigen on the target cell and activates the NFAT response element; and (b) detecting expression of the reporter protein, wherein detectable expression of the reporter protein indicates the presence of the target cell.

In some embodiments, the detected target cell is a cancer cell and the antigen-binding domain of the CAR binds a cancer cell-specific surface antigen on the target cell. In other embodiments, the detected target cell is a virus-infected host cell such as, for example, a Zika virus infected cell. In some such embodiments, the surface molecular antigen expressed on the virus-infected cell can be a Zika virus-specific envelope glycoprotein (Egp). For example, the antigen-recognizing portion of the CAR is modified or exchanged to quantitatively assess different viral pathogens such as dengue virus (DENY), West Nile (WNV), and Yellow Fever (YFV). In some embodiments, the methods harness the translational machinery of the infected host cell to process viral RNA into a virus-specific antigen that is detectable by the genetically engineered NK cell as described herein.

Some embodiments are directed to methods of treating or preventing a disease using genetically engineered NK cells expressing a CAR as a therapeutic agent. For example, provided herein are methods comprising administering a genetically engineered NK cell expressing the CAR as an active therapeutic agent. The disease against which the NK cell expressing the CAR is administered is not particularly limited as long as the disease shows sensitivity to the NK cell. Examples of the disease include a cancer (e.g., blood cancer (leukemia), solid tumor), an inflammatory disease/autoimmune disease (e.g., asthma, eczema), hepatitis, and an infectious disease, the cause of which is a virus such as Zika virus, influenza, and HIV, a bacterium, or a fungus, for example, tuberculosis, methicillin-resistant Staphylococcus aureus (MRSA), vancomycin-resistant enterococci (VRE), and deep mycosis. In some embodiments, a genetically engineered NK cell expressing the CAR binds to an antigen expressed on the surface of a target cell that targeted to be decreased or eliminated for treatment of the aforementioned diseases, that is, a tumor antigen, a viral antigen, a bacterial antigen or the like, is administered to treat or prevent such diseases. The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, methods described herein can provide any amount of any level of treatment or prevention of cancer in a mammal. Furthermore, the treatment or prevention provided by example methods can include treatment or prevention of one or more conditions or symptoms of the disease, e.g., cancer, being treated or prevented. Also, for purposes herein, “prevention” can encompass delaying the onset of the disease, or a symptom or condition thereof.

In some embodiments, genetically engineered NK cells are administered to a host (e.g., subject) in need thereof as a composition comprising the genetically engineered NK cells and a suitable carrier, diluent, or excipient as described herein. Any appropriate method of providing modified CAR-expressing cells to a host can be used for methods described herein. In some embodiments, methods for providing NK cells to a host can be adapted from clinical protocols for cellular and adoptive immunotherapy for infusion of donor-derived immune cells into a human host. In some embodiments, an adapted clinical protocol suitable for methods provided herein comprises obtaining NK cells from a host, genetically engineering (e.g., modifying) NK cells to express a CAR and NFAT-RE regulated protein transgene as described herein, and infusing the genetically engineered NK cells back into the host. A host, as used herein, includes and/or refers to any organism, such as a human, an animal (e.g., mammal, reptile, bird), insect, plant, among others, and which can be a subject of a study or test and/or a patient.

Administration of the genetically engineered NK cells provided herein can be administered by any appropriate route, including, without limitation, administration intravenously, intratumorally, intramuscularly, subcutaneously, intraperitoneally, intra-arterially, or into an afferent lymph vessel, by parenteral administration, for example, by injection or infusion. In some embodiments, where genetically engineered NK cells or populations of such NK cells are administered, the NK cells can be cells that are allogeneic or autologous to the host, such as a mammal. Preferably, the NK cells are autologous to the host.

In some embodiments, a host to which genetically engineered NK cells are provided is monitored or assessed for increased (e.g., improved, more robust) tumor clearance. Accordingly, various embodiments are directed to methods used for cancer therapies. In some embodiments, a host to which genetically engineered NK cells are provided is monitored or assessed for clearance of cells expressing a particular antigen.

Some embodiments are directed to a method for cell-based treatment or prevention against a pathogen of interest. For such methods, the genetically engineered NK cell comprises a polynucleotide sequence encoding a therapeutic protein place of, or in addition to, the polynucleotide sequence encoding the detectable reporter protein; and is fused with a signal peptide (sec) on the 3′ end of the polynucleotide sequence to assist in extracellular transport. Upon triggering the cascade effector NK cell activation events and activation of the NFAT response element, expression of a therapeutic protein is induced. The method can include the localized production of a therapeutic protein at the site of the target cell (e.g., a tumor cell, infected cell) and extracellular secretion of the therapeutic protein in the disease microenvironment.

Some embodiments are directed to methods for using genetically engineered NK cells as a sensor technology in a variety of applications. By way of example, transfusion-mediated spread of emerging flavivirus pathogens, e.g., Zika virus (ZIKV), dengue virus (DENY), has been identified as a serious risk. In order to protect donated blood supply, screening of donors that includes blood testing has been recommended. Clinical symptoms manifest in only 20% of ZIKV infections, and there are no reliable commercially available ZIKV diagnostic test kits for use outside the clinical laboratory. Identifying the infection is therefore challenging, especially given the similarity of symptoms with those of other diseases and the cross-reactivity of antibodies with other arboviruses (e.g., dengue, chikungunya). Accordingly, provided herein is a method comprising contacting a genetically engineered NK cell comprising a CAR having an antigen binding domain for detection and binding to an antigen specific to the virus of interest to a sample comprising or suspected of comprising cells infected with the virus of interest, and NFAT-RE regulated reporter transgene to inform the presence of the cells infected with the virus of interest.

Additional applications of the genetically engineered NK cells described herein include the following:

To target anticancer chemotherapeutic prodrugs to a tumor location, genetically engineered NK cells can be loaded with enzymatically activatable prodrugs, where the drug-activating enzyme is synthesized only at the tumor location, thus providing localized transformation of the prodrug into its active form. In some embodiments, the prodrug may not be loaded into the NK cells, and may be infused in multiple doses subsequent to the infusion of the genetically engineered NK cells. The prodrug can alternatively be bound to an imaging nanoparticle or other means of image-guided means of active drug delivery. Attaching the prodrug to an imaging nanoparticle or engineering the NK cells to express imaging transgenes enables the engineered NK cells to guide appropriate staging of the patient in preparation of surgery and for visually identifying and/or imaging tumor margins to assist in cytoreductive surgery.

Some embodiments are directed to methods of localized delivery of a chemotherapeutic agent to a site of the disease (e.g., tumor mass, site of autoimmune disease) comprises contacting a genetically engineered NK cell to a host cell population, wherein the genetically engineered NK cell comprises (i) an exogenous polynucleotide sequence encoding a CAR comprising an antigen binding domain, a transmembrane domain, and an intracellular signaling domain; and (ii) a NFAT response element operably linked to a polynucleotide sequence encoding an enzyme, wherein, in the presence of the target host cell in the contacted cell population, the genetically engineered NK cell binds to a surface molecular antigen on the target host cell and activates the NFAT response element to initiate expression of the enzyme, which acts on the prodrug predesigned to be activated by this enzyme and uses it membrane permeability due to its hydrophobicity to be released at the site of the disease.

With regard to surgical interventions for treating cancer, genetically engineered effector NK cells can be used for visualizing and/or imaging tumor margins via the expression of detectable reporter protein such as fluorescent proteins (e.g., GFP, GFP variants) or bioluminescent enzymes (e.g., luciferase). For example, such genetically engineered NK cells can be used to mark tumor margins to aid in surgical excision and to identify any residual positive tumor margins.

In some embodiments, genetically engineered NK cells are used for non-invasive detection and imaging of tumors based on expression of an imaging enzyme (e.g., thymidine kinase is capable of trapping a radioactive probe or otherwise detectable probe; tyrosinase detected by photoacoustic imaging or magnetic resonance imaging) expressed when tumor-specific CAR NK cells engage the antigen on tumor cells.

In some embodiments, genetically engineered NK cells can be used to circumvent safety concerns associated with vaccines against flaviviruses. By way of example, antigenic diversity among the four different dengue virus serotypes is responsible for the lack of antibody-mediated immunity and allows for multiple sequential infections. Although antibodies are effective in primary infection, their sub-neutralizing level during the secondary infections has been found to exacerbate the hemorrhagic fever by activating the complement system against the large infected cell mass in acute-phase. Prior dengue infection has also been found to worsen Zika infection. The use of NK cells can circumvent these safety concerns with flaviviruses because the NK cells, as described herein, can be engineered to express an antiviral protein, from human or non-human or synthetic origin, upon detecting the viral E glycoprotein (Egp) expressed on the surface of cells infected by the virus.

In some embodiments, genetically engineered NK cells comprise a CAR that detects a cancer-specific antigen on a target cancer cell (e.g., a HPV E6 or E7 antigen in case of cervical cancer) and a NFAT-RE to drive the expression of a reporter protein as described above. Such embodiments can be used for early detection of cancer.

In some embodiments, the genetically engineered NK cells comprise a CAR that detects an antigen on a pathogen-infected cell (e.g., detecting a ZIKV or DENY E glycoprotein on Zika- or dengue virus-infected cell) and a NFAT response element to induce expression of a reporter polypeptide. Such embodiments can be used for transfusion medicine to detect the presence of emerging pathogens (e.g. Zika, dengue, West Nile, Yellow Fever).

Different CARs can be used in genetically engineered NK cells with NFAT-RE regulated reporters to detect and measure signal-to-noise ratio to guide the selection of appropriate CARs for a cell-based therapy that exert the intended therapeutic effect without exhibiting unintended side-effects.

Mammalian cells can be engineered as NK cells to comprise a glucose-sensing GPCR (GPR1) which mobilizes internal Ca′ stores and NFAT response element-regulated to express engineered insulin. Such engineered NK cells can be used for autonomous synthesis of insulin upon sensing glucose. Such embodiments can be used for beta-cell replacement therapy.

Other non-limiting example uses of the genetically engineered NK cells include: i) imaging of the location of disease microenvironments to assist in surgical resection or monitor disease progression/regression; ii) cytotoxicity to kill the disease cells; iii) proliferation to enhance T-cell persistence; iv) immune-stimulation to recruit other immune cells; v) chemokine to recruit other immune cells; vi) immunosuppression to create localized immunosuppressive microenvironment; and vii) regeneration to enhance tissue healing.

As used herein, a target cell (sometimes herein interchangeably referred to as a “target cell of a host”, “target cell of interest”, “a diseased cell”, or “a target disease cell”) includes and/or refers to a cell of interest associated with a living organism (e.g., a biological component of interest). An antigen of the target cell includes and/or refers to a structure (e.g., binding site) of the target cell which the antigen binding domain of the receptor element can bind to (e.g., has an affinity for). The NK cell can be from a variety of different type of cells, such as human and non-human cells, and sometimes herein referred to as “the source”. As used herein, the terms “genetically modified” and “genetically engineered” are used interchangeably and include and/or refer to a prokaryotic or eukaryotic cell that includes an exogenous polynucleotide, regardless of the method used for insertion. In some embodiments, the NK cell is modified to comprise a non-naturally occurring nucleic acid molecule that is created or modified by the hand of man (e.g., using recombinant deoxyribonucleic acid (DNA) technology) or is derived from such a molecule (e.g., by transcription, translation, etc.). An NK cell that contains an exogenous, recombinant, synthetic, and/or otherwise modified polynucleotide is considered to be a genetically engineered NK cell.

“Nucleic acid”, as used herein, includes and/or refers to a “polynucleotide,” “oligonucleotide,” and “nucleic acid molecule,” and generally means a polymer of DNA or ribonucleic acid (RNA), which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In some embodiments, the nucleic acid does not comprise any insertions, deletions, inversions, and/or substitutions. However, it may be suitable in some instances, as discussed herein, for the nucleic acid to comprise one or more insertions, deletions, inversions, and/or substitutions. In some embodiments, the nucleic acid can encode additional amino acid sequences that do not affect the function of the CAR and polynucleotide and which may or may not be translated upon expression of the nucleic acid by a host cell.

Nucleic acids can be obtained using any suitable method, including those described by Maniatis et al., Molecular Cloning: A Laboratory Manual, Cold Spring Harbor, N.Y., pp. 280-281 (1982) and/or U.S. Patent Application Publication No. US2002/0190663, each of which are herein fully incorporated in their entireties for their teachings. Nucleic acids obtained from biological samples typically are fragmented to produce suitable fragments for analysis.

Nucleic acids and/or other moieties can be isolated. As used herein, “isolated” includes and/or refers to separate from at least some of the components with which it is usually associated whether it is derived from a naturally occurring source or made synthetically, in whole or in part. Nucleic acids and/or other moieties of the invention can be purified. As used herein, “purified” includes and/or refers s separate from the majority of other compounds or entities. A compound or moiety can be partially purified or substantially purified. Purity can be denoted by a weight by weight measure and can be determined using a variety of analytical techniques such as but not limited to mass spectrometry, HPLC, etc.

### EXPERIMENTAL EMBODIMENTS

A number of experimental embodiments were conducted to generate genetically engineered NK cells and to characterize the NK cells functionality. Example constructs used to generate genetically engineered NK cells include the nucleotide sequences set forth in SEQ ID NOS: 1-7. SEQ ID NOs: 1-7 are each synthetic DNA.

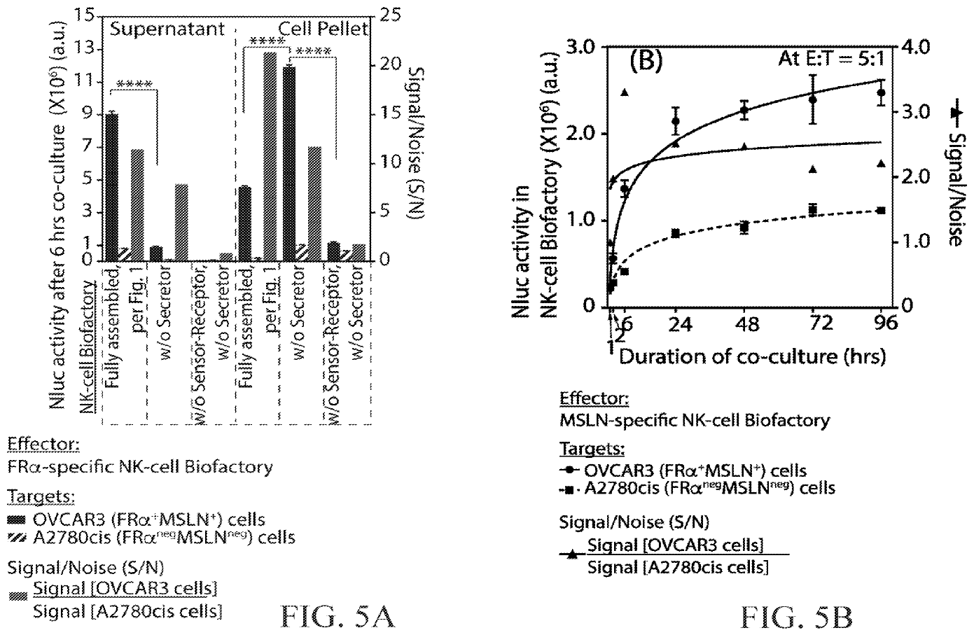

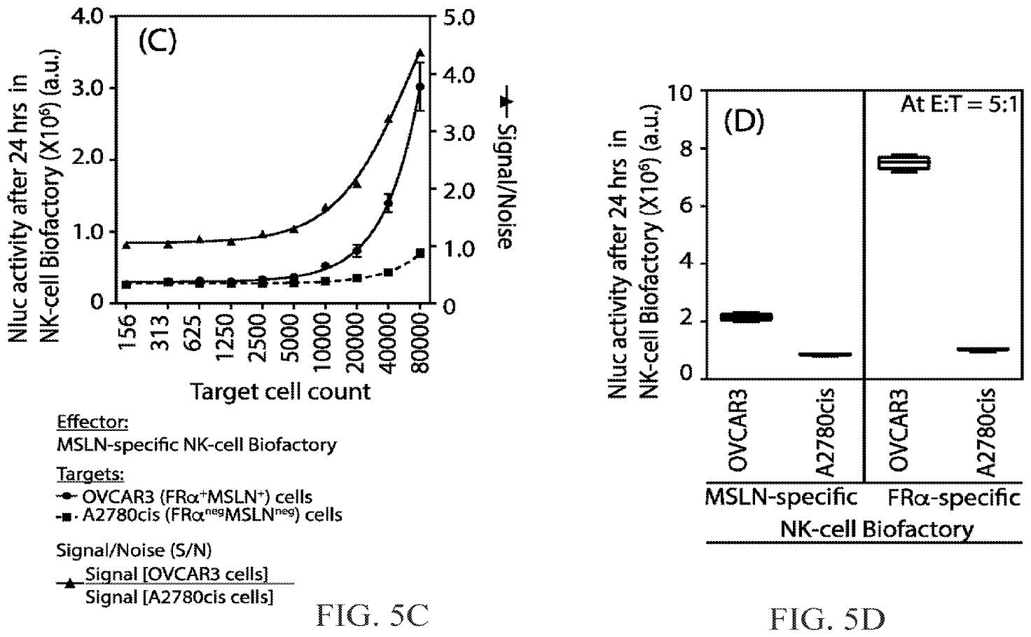

FIGS. 5A-5D illustrate example characterizations of genetically engineered NK cell function with specificity against FRα and MSLN antigens, in accordance with various embodiments. FRα and MSLN are antigens that are overexpressed on multiple human cancers and have limited expression on normal tissues. In experimental embodiments, OVCAR3 human ovarian cancer cells were used as target cells, which endogenously overexpress FRα and MSLN. A2780cis human ovarian cancer cells were used on non-target negative controls. A2780cis human ovarian cancer cells lack FRα and MSLN expression. For the extracellular antigen binding domain (sometimes referred to as the “sensor domain”), VH-VL sequences from the anti-FRα antibody (MORAb-003) and the anti-MSLN (MORAb-009) codon-optimized for human expressions were used. The NanoLuc® (Nluc) (Promega) reporter enzyme was used to represent an effector domain (e.g., the effector protein) that can be replaced by a human or non-human protein or peptide to induce autocrine and paracrine effect.

FIG. 5A illustrates the role the constant domains in antigen-directed functional response of the genetically engineered NK cell. The optimally configured artificial cell-signal pathway relates to the copies of the nuclear factor of the actuator element (e.g., the NFAT), as well as the intracellular co-stimulatory domains (e.g., the transmembrane domain, and an intracellular signaling domain) and the signal peptide. In experimental embodiments, the constant domains were combined with a FRα-specific binding domain to demonstrate that while the NK cell is non-responsive to FRα^{neg}MSLN^{neg}A2780cis cells, which are non-target cells, the NK cells synthesized and released the effector protein upon engaging to the target ligand on a surfaces of the FRa⁺MSLN⁺ OVCAR3 target cells. The signal-to-noise ratio (S/N), as defined in the legend, quantitates specificity of the genetically engineered NK cell. The function of the signal peptide for the release of the effector protein in the extracellular space was quantified by comparing the Nluc activity in the fully assembled genetically engineered NK cell and a control NK cell that lacked the signal peptide. The presence of signal peptide (e.g., a secretor) robustly correlated with the accumulation of Nluc in the supernatant (p<0.0001) and its absence was associated with Nluc build in the cell pellet (p<0.0001). Similarly, the function of the receptor element on regulating the artificial cell-signaling pathway and directing target-cell specificity (sensor or extracellular antigen binding domain that is part of the receptor element) was quantified by assessing the increased Nluc activity in the cell pellets of the two NK cells with and without the receptor element (p<0.0001).

FIG. 5B shows the kinetics of effector protein synthesis from the MSLN-specific NK cell. Nluc activity reporting effector synthesis increases as early as two hours (S/N of around two at two hours; S/N of around 3.3 at six hours, p<0.0001) when 12,500 MSLN-specific NK cells were stimulated by 2,500 OVCAR3 target cells. The activity stabilized within 24 hours and continued until at least 96 hours.

FIG. 5C shows the effector protein activity from the MSLN-specific NK cell as a function of the target cell mass (data collected at NK cell=12,500). While target cell-induced Nluc activity in the genetically engineered NK cell stimulated at low target/non-target count was not detectable, it increased exponentially with increasing target cell count and was statistically significantly different when stimulated by non-target cells beyond 2,5000 cell counts, e.g., E:T≤5:1 (p<0.05). This validates that the dose of the genetically engineered NK cells does not need to scale-up proportionate to the disease burden and indicates the genetically engineered NK cells can self-regulate the synthesis of the effector protein in situ.

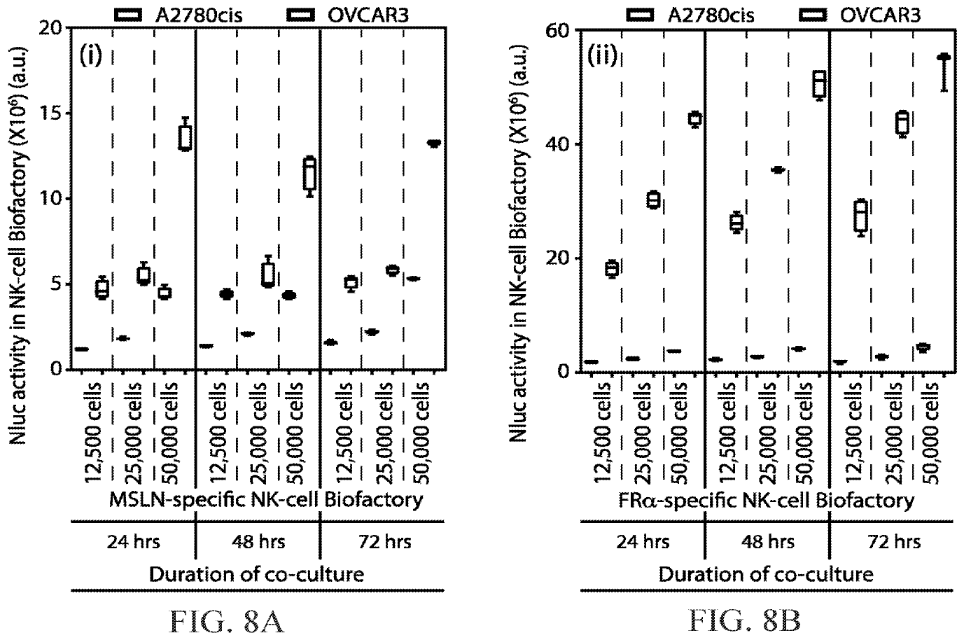

To show that the NK cell can be redirected to different target antigens, the sensor domain (e.g., the antigen binding domain) from the MSLN-specific scFV was exchanged with a FRα-specific scFV sequence. As shown by FIG. 5D, it was observed that the Nluc activity in the FRα-specific NK cell, when stimulated by the same number of OVCAR3 target cells over a period of 25 hours, was significantly upregulated compared to the MSLN-specific NK cells (12,500 effector cells and 2,500 target cells, e.g., E:T=5:1). This is similar to and higher than MSLN-specificity and FRα-specificity observed for modified T-cells, and may be due to higher expression of FRα on OVCAR3 cells, compared to MSLN expression. Alternatively, it may be due to higher integration of the FRα-specific artificial cell-signally pathway in the genetically engineered NK cell, compared to the integration of the MSLN-specific artificial cell-signaling pathway. Additional validations at 24, 48, and 72 hours with different E:T is further shown by FIGS. 8A-8B.

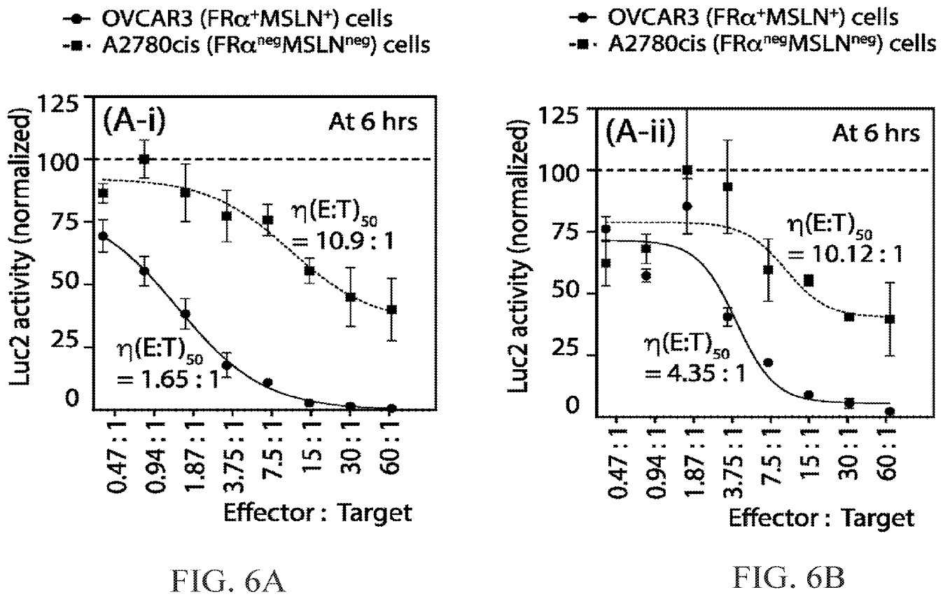

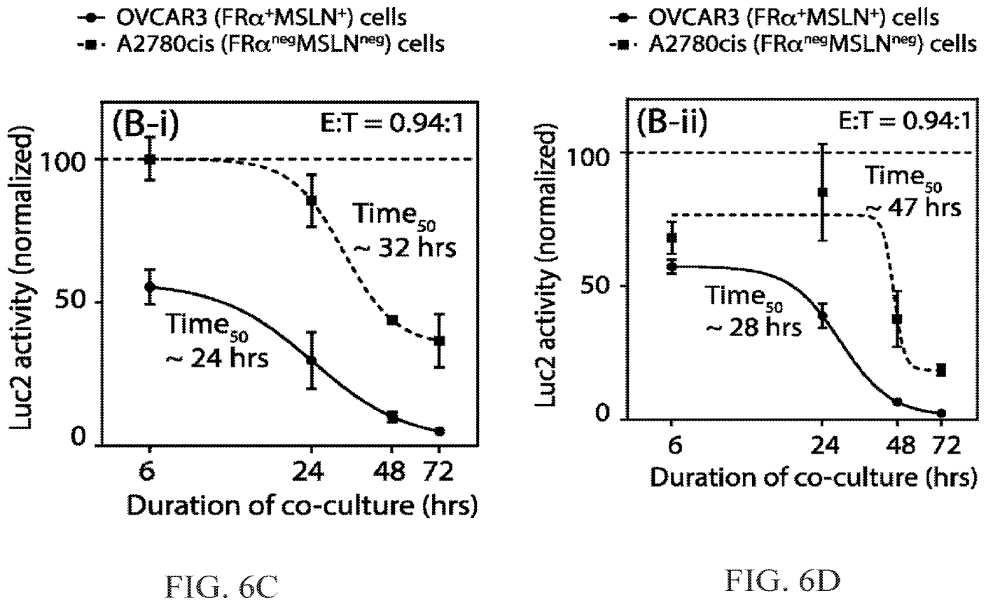

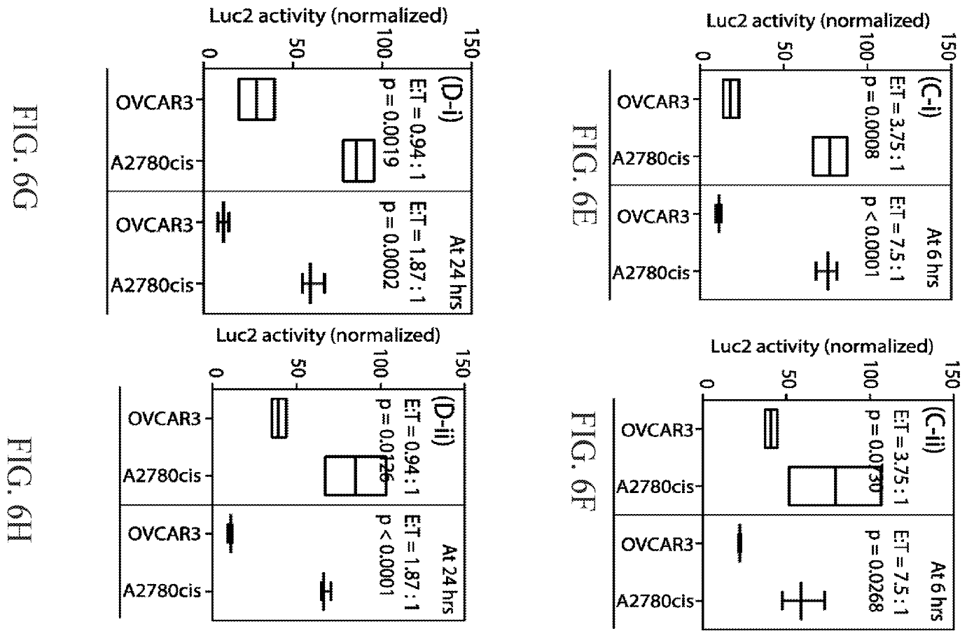

FIGS. 6A-6H illustrate example cytolytic function of MSLN-specific and FRα specific genetically engineered NK cells against target and non-target cells, in accordance with the present disclosure. More particularly, shown is the cytolytic function of MSLN-specific and FRα-specific NK cells against FRα⁺MSKB⁺ OVCAR3 target cells and FRα^{neg}MSLN^{neg}A2780cis non-target cells. The target and non-target cells were engineered to express Luc2® (firefly luciferase) (Promega), a 60.6-kDA, ATP-dependent bioluminescent reporter for in vitro and in vivo cell viability.

FIGS. 6A and 6B show the cytolytic activity of MSLN-specific NK cells (e.g., FIG. 6A) and FRα-specific NK cells (e.g., FIG. 6B) at 6 hours co-culture with OVCAR target cells, which was statistically significantly higher compared to that against non-target cells (FIG. 6A, shows p<0.05 at all E:T above 0.94:1 and FIG. 6B shows p<0.05 at all E:T above 3.75:1). A parameter for defining the target specific cytolytic efficiency, η(E:T)₅₀, was determined as the E:T at which Luc2 activity in target or non-target cells was 50% of the difference between the maximum and minimum values of their respective normalized Luc2 activities, when co-cultured with the genetically engineered NK cells for 6 hours. The η(E:T)₅₀ of the non-target A2780cis cells was higher (around 6.5-fold for MSLN-specific NK cells and around 2.5 for FRα-specific NK cells) than that of the target OVCAR3 cells.

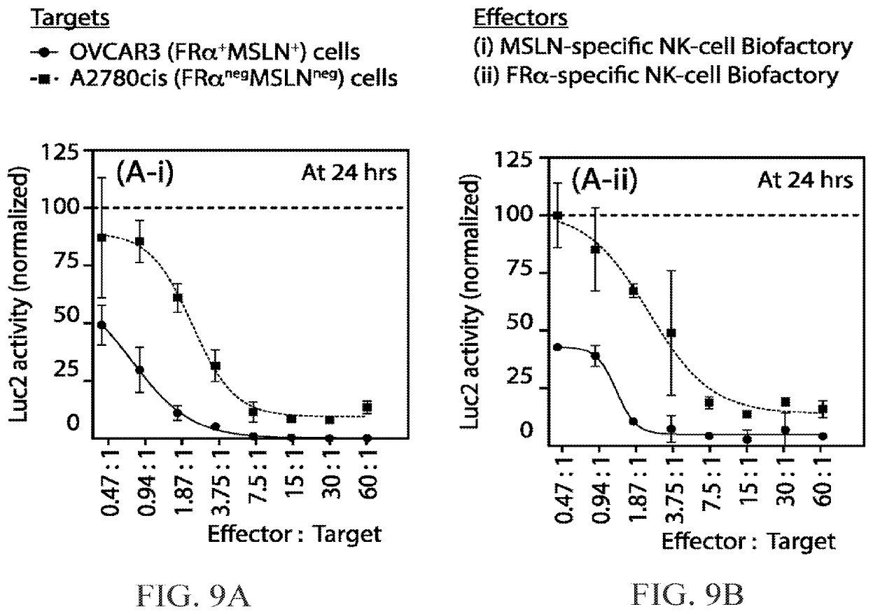

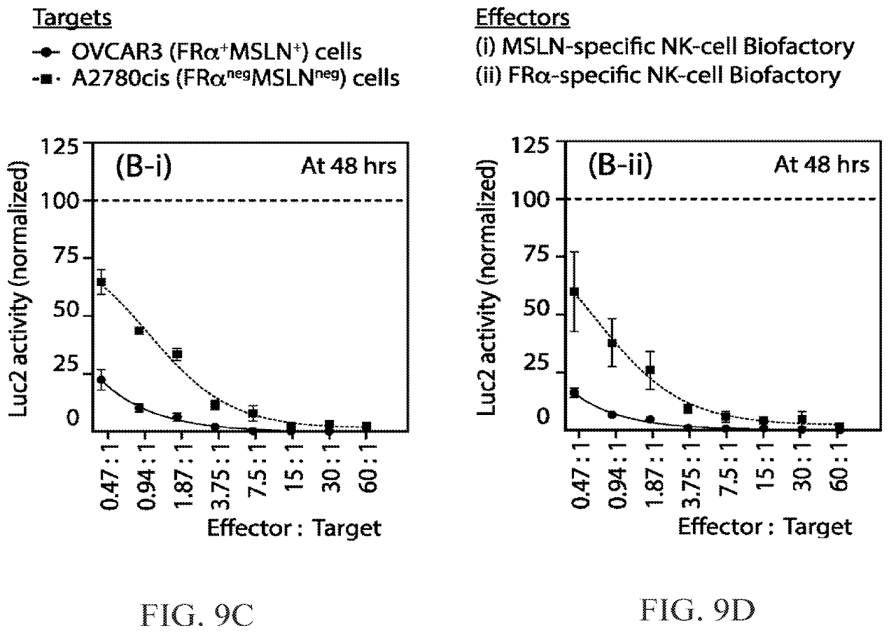

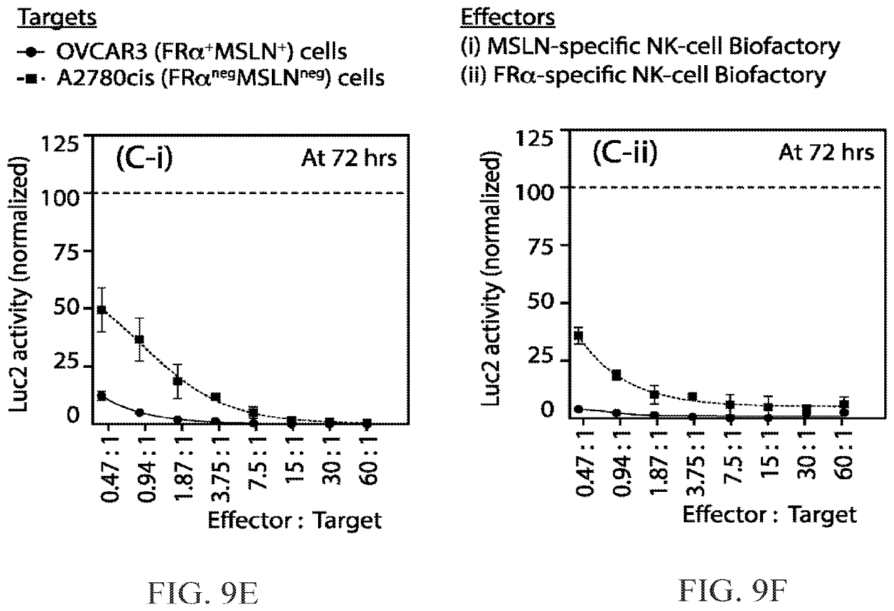

FIGS. 6C and 6D show the cytolytic activity of the MSLN-specific NK cells (e.g., FIG. 6C) and the FRα-specific NK cells (e.g., FIG. 6D) as a function of time and at E:T=0.94:1. The OVCAR (target) cell killing was again statistically higher compared to that of the A2780cis (non-target) cells (FIGS. 6C and 6D p<0.05 at all co-culture periods). Time₅₀ was determined as the duration of stimulation at which Luc2 activity in target or non-target cells was 50% of the difference between the maximum and minimum values of their respective normalized Luc2 activities, when co-cultured with the NK cells at E:T=0.94:1 (Note that Luc2 activity was normalized at each time point, where 100%=no NK cell (negative control) used and 0%=0.5% Tween20 (positive control)). The difference between the means became more pronounced at 24 hours and diminished again over longer durations, which allowed for the cumulative increase in non-specific cytolytic activity. This effect is further shown by FIGS. 9A-9F.

FIGS. 6E-6F and 6G-6H show the cytolytic activity of the MSLN-specific NK cells (e.g., FIG. 6E, 6G) and the FRα-specific NK cells (e.g., FIG. 6F, 6H) at different E:T at 6 and 24 hours co-culture with OVAR3 (target) cells and A2780cis (non-target) cells, respectively. A lower E:T demonstrated sufficient differential cytolytic effect at longer co-culture periods, an effect that has also been observed in vivo. Therefore, E:T along with the duration over which the target-specific cytolytic effect is observed, should be carefully balanced when designing in vitro assays. Additional validations with a larger E:T range are shown below.

The NK-92MI is a clinically relevant cell line and the cells are irradiated at 10 gray (Gy) before human infusion. This stopped further proliferation and rendered the cells non-oncogenic. Up to 10 billion NK cells/m2 have been safely infused in humans with no severe side effects. However, the resulting DNA damage also has the potential to disrupt actuator and effector element sequences, presenting a risk for the genetically engineered NK cell to be non-functional.

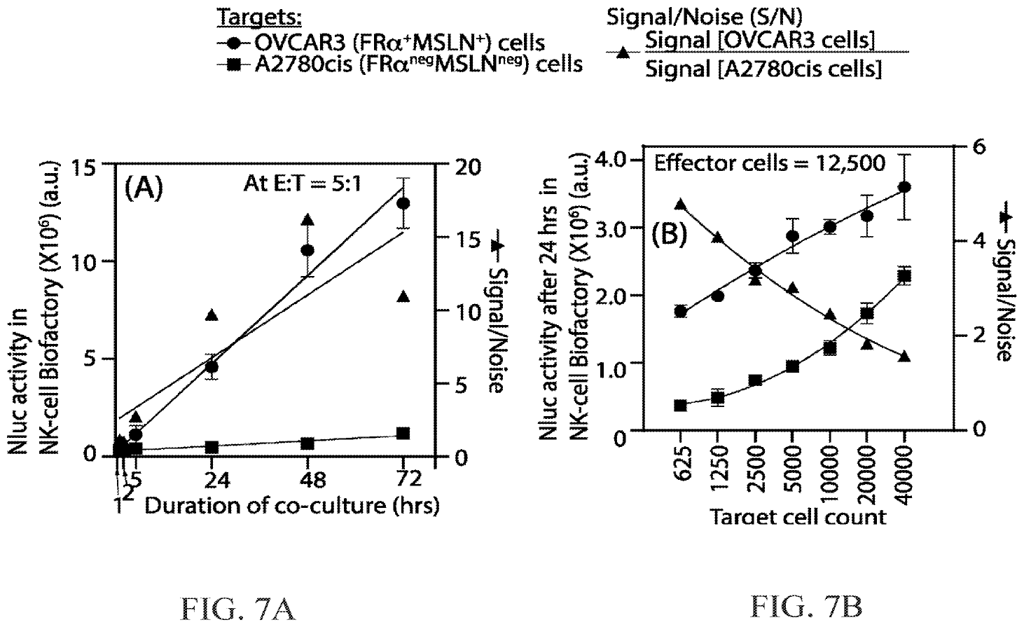

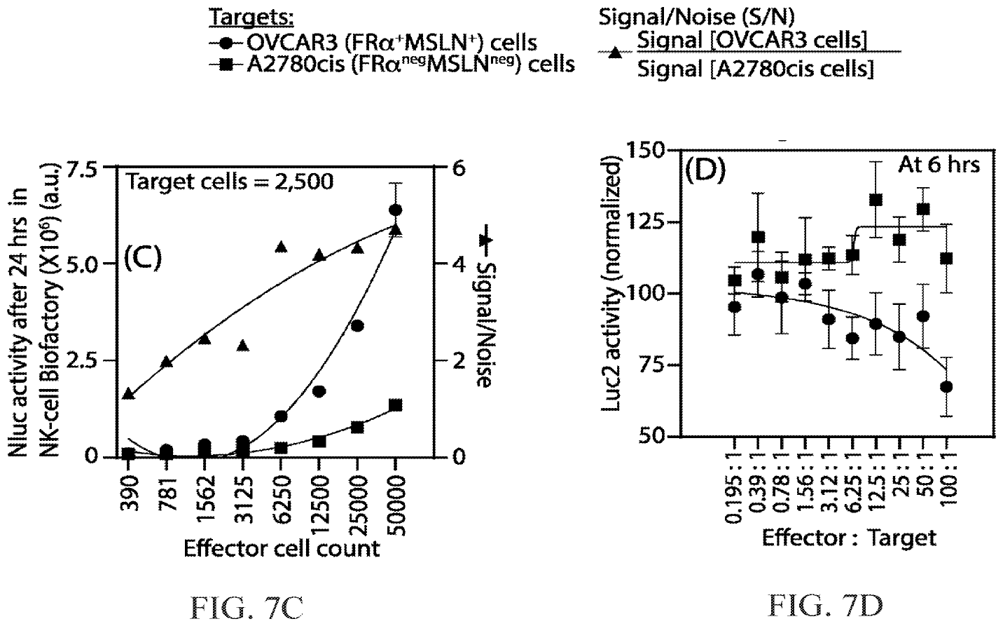

FIGS. 7A-7D illustrate the artificial cell-signaling pathway of example genetically engineered NK cell, in accordance with the present disclosure. FIGS. 7A-7D demonstrate that the integrity of the artificial cell-signaling pathway, and ultimately that of the genetically engineered NK cell, is preserved after exposure to 15 Gy radiation, thereby making it safe for clinical use.

FIG. 7A shows the kinetics of effector protein synthesis from FRα-specific NK cells. Nluc activity reporting effector protein synthesis increased at 5 hours (S/N around 3 at 5 hours, p<0.02) when stimulated by OVCAR3 (target) cells as compared to A2780cis (non-target) cells (12,500 NK cell and 2,500 target cell, e.g., E:T=5:1), and increases exponentially until at least 72 hours.

FIG. 7B shows the effector protein activity from the irradiated FRα-specific NK cells as a function of the target cell mass. The target cell induced Nluc activity in the 12,5000 NK cell, sometimes referred to as a biofactory, was statistically elevated when stimulated by OVCAR3 (target) cells compared to A2780cis (non-target) cells at cell counts (p=<0.50).

FIG. 7C shows the effector protein activity from the same irradiated FRα-specific NK cells as a function of the increase in their numbers with a constant target/non-target cell mass. The Nluc activity was statistically elevated when stimulated by 2,500 OVCAR3 (target) cells as compared to A2780cis (non-target) cells at all cell counts beyond E:T=0.625:1 (1562 NK cell biofactory for 2,500 target/non-target cells) (p=<0.01). The dose 10 Gy has also been found to be acceptable in clinical trials in terms of their cytolytic efficacy.

FIG. 7D shows the results of the cytolytic assay conducted with the FRα-specific NK cells irradiated at 15 Gy. The irradiated NK cell biofactory was co-cultured for 6 hours with OVAR3 (target) cells, resulting in a statistically significant increases of target-specific (OVCAR3) cytolysis (p<0.05 at E:T=l₀₀:1) compared to that against A2780cis (non-target) cells.

Synthesis and Experimental Information

(1) Materials and reagents. Lentivirus particles were prepared in HEK293T/17 (ATCC, Cat #CRL-11268) producer cells cultured in complete DMEM [DMEM growth media (Corning, Cat #10-013-CV) supplemented with 10% heat-inactivated fetal bovine serum (FBS) (Sigma-Aldrich, Cat #F2442-500ML) and 1X Penicillin Streptomycin solution (Corning, Cat #30-002-Cl)]. Plasmid transfections were performed using Transporter 5™ reagent (Polysciences, Inc, Cat #26008-5). Plasmids encoding different genetic payloads (transfer plasmids) were designed in SnapGene software (GSL Biotech LLC) and sub-cloned into lentivirus vector plasmid (System Biosciences, Cat #CD510B-1). Plasmids encoding 2^{nd} generation packaging plasmids (psPAX2—Cat #12260, pMD2.G— Cat #12259) were obtained from Addgene. pAdvantage was obtained from Promega (Cat #E1711). Plasmid preparation services (chemical synthesis of DNA insert sequences, sub-cloning into respective vector backbones, and the amplification) were obtained from Epoch Life Science, Inc. (Missouri City, TX). NK-92MI (ATCC, Cat #CRL-2408) cell line was maintained in compete NK-02MI media [RPMI1640 (Corning, Cat #10-040-CV), 20% FBS (Sigma-Aldrich, Cat #F2442-500ML), 1X GlutaMAX solution (Gibco, Cat #35050-061), and 1X Penicillin Streptomycin solution (Corning, Cat #30-002-CI)]. OVCAR3 (ATCC, Cat ##HTB-161) and A2780cis (Sigma-Aldrich, Cat #93112517) cell lines were maintained in complete RPMI [RPMI1640 (Corning, Cat #10-040-CV), 10% FBS (Sigma-Aldrich, Cat #F2442-500ML), and 1X Penicillin Streptomycin solution (Corning, Cat #30-002-Cl)]. Phosphate buffered saline (PBS) without Ca⁺² and Mg⁺² (Corning, Cat #21-040-CV) was used to minimize cell clumping. Puromycin N-acetyltransferase was used as a selection marker in all cases. Puromycin dihydrochloride (Puromycin) (ThermoFisher Scientific, Cat #A1113803) was used for selecting stable cell lines. Freezing media comprised of 50% heat-inactivated FBS (Sigma-Aldrich, Cat #F2442-500ML), 40% RPMI1640 (Corning, Cat #10-040-CV) and 10% DMSO (Sigma-Aldrich, Cat #D2650) for maintaining liquid nitrogen stocks of cell lines.

(2) Lentivirus production. Lentivirus particles were prepared by packaging the corresponding transfer plasmid using 2^{nd} generation lentivirus system as detailed by Radhakrishnan H, Javitz H S, Bhatnagar P, entitled “Lentivirus Manufacturing Process for Primary T-Cell Biofactory Production”, Advanced Biosystems, 1900288 (2020), which is hereby incorporated herein in its entirety for its teaching.

(3) Generation of NK cell biofactory. The 92MI suspension cell line was engineered with lentivirus particles carrying the appropriate (fully assembled (see FIG. 2) or control) NK cell biofactory, such as described by Radhakrishnan H, Javitz H S, Bhatnagar P, Advanced Biosystems, 1900288 (2020). Briefly, the cells were treated with lentivirus in the presence of 8 μg/mL Polybrene (Abm®, Cat #G062). After 48 hours, cells were placed in selection using 0.5 μg/ml of puromycin dihydrochloride. The unmodified parental cell line was also placed under selection as a positive control for cell killing by puromycin. Following selection, cells were expanded as required for different assays and frozen using freezing media. In some experiments, there were differences between the fully assembled biofactory, as shown by FIG. 2, and a control biofactory plasmid, such as described by Repellin C E, Patel P, Beviglia L, Javitz H, Sambucetti L, Bhatnagar P, entitled “Modular Antigen-Specific T-cell Biofactories for Calibrated In Vivo Synthesis of Engineered Proteins”, Advanced Biosystems, 2(12):1800210 (2018), which is hereby fully incorporated herein in its entirety for its teaching.

(4) Generation of irradiated NK cell biofactory. The NK cell biofactory were irradiated at 15 Gy using a ¹³⁷Cs γ-emitting irradiator, Mark I-68A (JL Shepherd and Associates) at a dose rate of 222 mGy/minute. The control groups were treated similarly except for the radiation.

(5) Co-culture of NK Cell (E) [NK cell biofactory (MSLN-specific or FRα-specific)] with Target-Cell (T) [Parental OVCAR3 or FRα⁺MSLN⁺Luc2-2A-E2Crimson⁺ OVCAR3 (target) cells or parental A2780cis or FRanegMSLNnegLuc2-2A-E2Crimson⁺ A2780cis (non-target) cells]. Target OVCAR3 or non-target A2780cis cells were co-cultured at different NK cell-to-Target-Cell ratio (E:T) ratios with MSLN-specific or FRα-specific NK cell biofactory in 100 μL of complete NK-92MI media in a single well of a 96-well plate. After the specified amount of time in co-culture, the manufacturer's protocol was followed to measure the reporter activity, e.g., NanoLuc® (Nluc) activity in the NK cell biofactory using Nano-Glo® assay (Promega, Cat #N1120) or Luc2 activity in A2780cis and OVCAR3 cells using One-Glo® assay (Promega, Cat #E6110). Briefly, the enzyme substrate (Nluc substrate or Luc2 substrate) was diluted in the cell lysis buffer provided with the Nano-Glo® or One-Glo® assay and added to the co-cultures in 96-well plate for assessing enzyme (Nluc or Luc2) activity, respectively. Following a brief incubation period (3 minutes for Nluc or 10 minutes for Luc2), bioluminescence was read on a microplate reader (Perkin Elmer, EnVision™ Multilabel Plate Reader Model: 2104-0010A).

(6) Signal peptide experiment and co-culture. The three differently engineered NK cell biofactory per FIG. 2 [(i) fully assembled (FRα-specific); (ii) without signal peptide or the secretor domain (FRα-specific); and without the signal peptide and receptor element domains] (125,000 cells) were washed with serum-free RPMI and resuspended in 200 μL of fresh complete NK-92MI media. Parental OVCAR3 (FRα⁺MSLN⁺ target cell line) and A2780cis (FRα^{neg}MSLN^{neg} non-target cell line) cells were seeded at 250,000 cells/well and similarly treated and resuspended in 200 μL of fresh complete NK-92MI media. The three NK cell biofactories were mixed with OVCAR3 target or A2780cis non-target cells to create six individual co-cultures in a 24-well plate of 400 μL and the final volume was made up to 1 mL with complete NK-92MI media. After 6 hours, a 500-μL aliquot from each co-culture was centrifuged in 1.5-mL tube for 5 min at 200 RCF.
a. Effector Nluc assessment in supernatant. 100 μL of supernatant was drawn without disturbing the cell pellet and transferred to a 96-well plate (4 wells were prepared for every co-culture). The Nluc activity was assessed in a 96-well plate with Nano-Glo® assay using the manufacturer's protocol.
b. NK cell Nluc assessment in cell pellet. Exactly 1 mL of complete NK-92MI media was added to the remaining 100 μL in each 1.5-mL tube (contains cell pellet). Cell pellets were washed three times by removing and adding exactly 1 mL of complete NK-92MI media each time. Exactly 400 μL of complete NK-92MI media was added to the remaining 100 μL in the 1.5-mL tube and the cell pellets were resuspended back in total of exactly 500 μL of complete NK-92MI media. Exactly 100 μL of resuspended cells were drawn from each 1.5-mL tube and transferred to a 96-well plate (4 wells were prepared for every co-culture). The Nluc activity was assessed in the 96-well plate with Nano-Glo® assay using the manufacturer's protocol.

(7) Experimental designs and statistical analysis. GraphPad Prism 8.1.1 (GraphPad Software, Inc.) was used to conduct all statistical analysis. Statistical methods for all group comparisons and fitted equations are reported below. The following provides additional detail for the above described figures.

FIGS. 5A-5D (Engineered function of the NK cell biofactory). The statistical analysis for FIGS. 5A and 5D were based on two-sample t-tests [unpaired student's two-tailed t-test] with common variance and a two-sided p-value of 0.05. There was no adjustment for multiple comparisons. Analyses for FIG. 5B and FIG. 5C were based on an ANOVA followed by the two-stage linear step-up procedure of Benjamini, Krieger, and Yekutieli with a false discovery rate of less than 1%. The S/N is calculated as the ratio of the mean Nluc activity in the NK cell biofactory when stimulated by the parental OVCAR3 (FRα⁺MSLN⁺) cells divided by the mean Nluc activity when stimulated by the parental A2780cis (FRαnegMSLNneg) cells. The error bars extend 1 SD above and below the mean and can also be considered as one half-width of a 68% confidence interval for that mean.

FIG. 5A (Functional components of the NK cell biofactory). The vertical bars show the mean Nluc activity in three differently engineered FRα-specific NK cell biofactories when stimulated by the target OVCAR3 (FRα⁺MSLN⁺) or non-target A2780cis (FRα^{neg}MSLN^{neg}) cells. One NK cell biofactory was fully assembled (per FIG. 2), a second NK cell biofactory was a control that lacked the IFNα2 Secretor domain (per FIG. 2 but without the signal peptide), and a third NK cell biofactory was a control that lacked the IFNα2 signal peptide and the FRα-specific receptor element domain (per FIG. 2 but without the signal peptide and without the receptor element).

FIG. 5B (NK cell biofactory activates within 2 hours). The Nluc activity in MSLN-specific NK cell biofactory (per FIG. 2 but without the signal peptide) stimulated by the target OVCAR3 (FRα⁺MSLN⁺) or non-target A2780cis (FRα^{neg}MSLN^{neg}) cells was fit using the equation Y=a+b*log₁₀(X) where X is the stimulation time in hours.

FIG. 5C (N cell biofactory activates as a function of the target cell mass). The Nluc activity in the MSLN-specific NK cell biofactory (per FIG. 2 but without the signal peptide) stimulated by the target OVCAR3 (FRα⁺MSLN⁺) or non-target A2780cis (FRα^{neg}MSLN^{neg}) cells was fit using a four-parameter logistic model Nluc=Nlucₘᵢₙ+{Nlucₘₐₓ−Nlucₘᵢₙ}/{1+10\hat{ }[b*(log₁₀[Target₅₀]−X)]} where X is the log₁₀ of the target cell count, Nlucₘₐₓ is an estimated parameter defining a upper asymptote for Nluc activity, Nlucₘᵢₙ is an estimated parameter defining a lower asymptote for Nluc activity, b is a “Hill” parameter defining the slope at the inflection point of the fitted curve, and Target₅₀ is an estimated parameter representing the X value corresponding to (Nlucₘₐₓ−Nlucₘᵢₙ)/2.

FIG. 5D (NK cell biofactory can be redirected towards different cancer antigens). The Tukey box-and-whisker plot shows the Nluc activity in the two NK cell biofactories (FRα-specific receptor element using VH-VL sequence from MORAb-003 or MSLN-specific receptor element using VH-VL sequence from MORAb-009) stimulated by the target OVCAR3 (FRα⁺MSLN⁺) or non-target A2780cis (FRα^{neg}MSLN^{neg}) cells. Both NK cell Biofactories were generated per FIG. 2 with FRα-specific or MSLN-specific receptor element but without the signal peptide.

FIGS. 6A-6H (Innate cytolytic function the NK cell biofactory). Luc2 activity was normalized at each time point, where 100%=maximum Luc2 activity and 0%=Tween20 (complete cell killing). Statistical analysis for (A) and (B) were based on ANOVA followed by two-stage linear step-up procedure of Benjamini, Krieger, and Yekutieli with a false discovery rate of less than 1%. Analyses for (C) and (D) were based on two-sample t-tests [unpaired student's two-tailed t-test] with common variance and a two-sided p-value of 0.05. There was no adjustment for multiple comparisons. The error bars extend 1 SD above and below the mean and can also be considered as one half-width of a 68% confidence interval for that mean. All experiments included both NK cell biofactories (FRα-specific receptor element using VH-VL sequence from MORAb-003 and MSLN-specific receptor elements using VH-VL sequence from MORAb-009) stimulated by the FRα⁺MSLN⁺Luc2-2A-E2Crimson⁺ OVCAR3 (target) cells or FRα^{neg}MSLN^{neg}Luc2-2A-E2Crimson⁺ A2780cis (non-target) cells. Luc2 activity in target or non-target was assessed as a surrogate biomarker for live cells.

FIGS. 6A-6B (Cytolytic activity of the two NK cell biofactories at 6 hours with respect to the number of NK cell biofactory). The normalized Luc2 activity when NK cell biofactory (per FIG. 2 but without the signal peptide) was co-cultured with Luc2⁺ target OVCAR3 (FRα⁺MSLN⁺) or non-target A2780cis (FRα^{neg}MSLN^{neg}) cells was fit using a four-parameter logistic model, Luc2=Luc2 min+{Luc2max−Luc2 min}/{1+10 \hat{ }[b*(log₁₀[η(E: T)₅₀]−X)]}.
i) where X is the log₁₀ of the number of NK cells, sometimes herein referred to as effector-cells or NK-cell biofactories, Luc2ₘₐₓ is an estimated parameter defining an upper asymptote for Luc2 activity, Luc2ₘᵢₙ is an estimated parameter defining a lower asymptote for the Luc2 activity, b is a “Hill” parameter defining the slope at the inflection point of the fitted curve; and
ii) a parameter for defining the target-specific cytolytic efficiency, η(E:T)₅₀, was determined as E:T at which Luc2 activity in target or non-target cells was 50% of the difference between the maximum and minimum values of their respective normalized Luc2 activity, when co-cultured with the NK cell biofactory, e.g., the η(E:T)₅₀ is an estimated E:T value corresponding to (Luc2ₘₐₓ−Luc2ₘᵢₙ/2).

FIGS. 6C-6D (Cytolytic activity of the two NK cell biofactories at E:T of 0.94:1 with respect to the duration of NK cell biofactory stimulation). The normalized Luc2 activity with NK cell biofactory (per FIG. 2 but without the signal peptide) was co-cultured with Luc2+ target OVCAR3 (FRα⁺MSLN⁺) or non-target A2780cis (FRα^{neg}MSLN^{neg}) cells was fit using a four-parameter logistic model, Luc2=Luc2ₘᵢₙ+{Luc2ₘₐₓ−Luc2ₘᵢₙ}/{1+10\hat{ }[b*(log¹⁰[Time₅₀]−X)]},
i) where X is the login of the duration of NK cell biofactory stimulation, Luc2ₘₐₓ is an estimated parameter defining an upper asymptote for Luc2 activity, Luc2ₘᵢₙ is an estimated parameter defining a lower asymptote for the Luc2 activity, b is a “Hill” parameter defining the slope at the inflection point of the fitted curve; and
ii) Times₅₀ was determined as the duration of stimulation at which Luc2 activity in target or non-target cells was 50% of the of the difference between the maximum and minimum values of their respective normalized Luc2 activities, when co-cultured with the NK cell biofactory, e.g., the Times₅₀ is an estimated duration value corresponding to (Luc2ₘₐₓ−Luc2ₘᵢₙ/2).

FIGS. 6E-6F (Cytolytic activity of the two NK cell biofactoroes at different E:T at 6 hour duration of NK cell biofactory stimulation). The Tukey box-and-whisker plot shows the normalized Luc2 activity when the two NK cell biofactories (FRα-specific or MSLN-specific) were co-cultured with the Luc2⁺ target OVCAR3 (FRα⁺MSLN⁺) or non-target A2780cis (FRα^{neg}MSLN^{neg}) cells for 6 hours. Both NK cell biofactories were generated per FIG. 2 with FRα-specific or MSLN-specific receptor element but without the signal peptide. E:T of 3.75:1 and 7.5:1 were used.

FIGS. 6G-6H (Cytolytic activity of the two NK cell biofactories at different E:T at 24 hours duration of NK cell biofactory stimulation). The Tukey box-and-whisker plot shows the normalized Luc2 activity when the two NK cell biofactories (FRα-specific or MSLN-specific) were co-cultured with the Luc2⁺ target OVCAR3 (FRα⁺MSLN⁺) or non-target A2780cis (FRα^{neg}MSLN^{neg}) cells for 24 hours. Both NK cell biofactories were generated per FIG. 2 with FRα-specific or MSLN-specific receptor element but without the signal peptide. E:T of 3.75:1 and 7.5:1 were used.

FIGS. 7A-7D (Engineered and innate function of the irradiated NK cell biofactory). Statistical analyses were based on multiple t-tests followed by two-stage linear step-up procedure of Benjamini, Krieger, and Yekutieli with a false discovery rate of 1%. There was no adjustment for multiple comparisons. The error bars extend 1 SD above and below the mean and can also be considered as one half-width of a 68% confidence interval for that mean. All experiments included irradiated (15 Gy) FRα-specific NK cell biofactory stimulated by the FRα⁺MSLN⁺Luc2-2A-E2Crimson⁺ OVCAR3 (target) cells or FRα^{neg}MSLN^{neg}Luc2-2A-E2Crimson⁺ A2780cis (non-target) cells. The S/N in (A), (B), and (C) is calculated as the ratio of the mean Nluc activity in the NK-cell Biofactory when stimulated by the parental OVCAR3 (FRα⁺MSLN⁺) cells divided by the mean Nluc activity when stimulated by the parental A2780cis (FRα^{neg}MSLN^{neg}) cells.

FIG. 7A (Irradiated NK cell biofactory activates within 5 hours). The Nluc activity in irradiated NK cell biofactory (per FIG. 2 but without the signal peptide) stimulated by the target OVCAR3 (FRα⁺MSLN⁺) or non-target A2780cis (FRα^{neg}MSLN^{neg}) cells was fit using the equation Y=a+b*X, where X is the stimulation time in hours. (Note: Engineered function of the non-irradiated NK cell biofactory is modeled based on a logarithmic curve. However, this function is reduced for the irradiated NK cell biofactory and can be explained using the linear regression model that represents the early part of the logarithmic curve).

FIG. 7B (Irradiated NK cell biofactory activates as a function of the target cell mass). The Nluc activity in the irradiated NK cell biofactory (per FIG. 2 but without the signal peptide) stimulated by the target OVCAR3 (FRα⁺MSLN⁺) or non-target A2780cis (FRα^{neg}MSLN^{neg}) cells was fit using a four-parameter logistic model Nluc=Nlucₘᵢₙ+{Nlucₘₐₓ−Nlucₘᵢₙ}/{1+10\hat{ }[b*(log₁₀[Target₅₀]−X)]}, where Nlucₘₐₓ is an estimated parameter defining an upper asymptote for Nluc activity, Nlucₘᵢₙ is an estimated parameter defining an lower asymptote for Nluc activity, is a “Hill” parameter defining the slope at the inflection point of the fitted curve, and Targets₅₀ is an estimated parameter representing the X value corresponding to (Nlucₘₐₓ−Nlucₘᵢₙ)/2.

FIG. 7C (Target cell induced function in the irradiated NK cell biofactory is proportional to cell count). The Nluc activity in the irradiated NK cell biofactory (per FIG. 2 but without the signal peptide) stimulated by the target OVCAR3 (FRα⁺MSLN⁺) or non-target A2780cis (FRα^{neg}MSLN^{neg}) cells was fit using a four-parameter logistic model Nluc=Nlucₘᵢₙ+{Nlucₘₐₓ−Nlucₘᵢₙ/{1+10\hat{ }[b*(log₁₀[Effector₅₀]−X)]}.

FIG. 7D (Innate cytolytic activity of the irradiated NK cell biofactory at 6 hours with respect to the number of NK cell biofactory). The normalized Luc2 activity, a surrogate marker for target cell cytolysis induced by irradiated NK cell biofactory (per FIG. 2 but without the signal peptide) when co-cultured with the Luc2⁺ target OVCAR3 (FRα⁺MSLN⁺) or non-target A2780cis (FRα^{neg}MSLN^{neg}) cells was fit using a four-parameter logistic model, Luc2=Luc2ₘᵢₙ+{Luc2ₘₐₓ−Luc2ₘᵢₙ}/{1+10\hat{ }[b*(log10[η(E:T)₅₀]−X)]},
i) where X is the login of the duration of NK cell biofactory stimulation, Luc2ₘₐₓ is an estimated parameter defining an upper asymptote for Luc2 activity, Luc2ₘᵢₙ is an estimated parameter defining a lower asymptote for the Luc2 activity, b is a “Hill” parameter defining the slope at the inflection point of the fitted curve; and
ii) a parameter for defining the target-specific cytolytic efficiency, η(E:T)₅₀, was determined as E:T at which Luc2 activity in target or non-target cells was 50% of the difference between the maximum and minimum values of their respective normalized Luc2 activity, when co-cultured with the NK cell biofactory, e.g., the η(E:T)₅₀ is an estimated E:T value corresponding to (Luc2ₘₐₓ Luc2ₘᵢₙ/2).

As further described below, statistical analysis for FIGS. 8A-8B were based on two-sample t-tests [unpaired student's two-tailed t-test] with common variance and two-sided p-value of 0.05. There was no adjustment for multiple comparisons. Statistical analysis for FIGS. 9A-9F were based on ANOVA followed by two-stage linear step-up procedure of Benjamini, Krieger, and Yekutieli with a false discovery rate of less than 1%. The error bars extend 1 SD above and below the mean and can also be considered to be one half-width of a 68% confidence interval for that mean.

FIGS. 8A-8B illustrate example of genetically engineered NK cells being redirected toward different cancer antigens, in accordance with the present disclosure. The Tukey box-and-whisker plots show the Nluc activity for the (i) MSLN-specific (FIG. 8A) and (ii) FRα-specific (FIG. 8B) NK cell biofactory stimulated by the target (FRα⁺MSLN⁺ OVCAR3) or non-target (FRα^{neg}MSLN^{neg}A2780cis) cells. Both NK cell biofactories were generated per FIG. 2 with FRα- or MSLN-specific receptor element but without the signal peptide. All data was collected at target/non-target cells=2,500. Different E:T were used, e.g., 20:1, 10:1, and 5:1; and Nluc activity was observed at 24, 48, and 72 hours, as indicated.

FIGS. 9A-9F illustrate cytolytic function of example genetically engineered NK cells, in accordance with the present disclosure. All experiments included both NK cell biofactories [(i) MSLN-specific receptor element (FIGS. 9A, 9C, 9E), and (ii) FRα-specific (FIGS. 9B, 9D, and 9F) receptor element] stimulated by either FRα⁺MSLN⁺Luc2-2A-E2Crimson⁺ OVCAR3 (target) cells or FRα^{neg}MSLN^{neg}Luc2-2A-E2Crimson⁺ A2780cis (non-target) cells. Both NK cell biofactories were generated per FIG. 2 with FRα- or MSLN-specific receptor element but without the signal peptide. Luc2 activity in target or non-target cells was assessed as a surrogate biomarker for live cells. In all experiments, 0.5% Tween-20 was used as positive control for cell killing. The cytolytic activity as a function of E:T was fit using a four-parameter logistic model at A) 24 hours (FIGS. 9A-9B), B) 48 hours (FIGS. 9C-9D), and C) 72 hours (FIGS. 9E-9F). All data was collected at target/non-target cells=2,500. Luc2 activity for all observations was measured using n=3. The error bars represent 1 SD above and below the mean and can also be considered as one half-width of a 68% confidence interval for that mean. The normalized Luc2 activity in NK cell biofactory (per FIG. 2 but without the signal peptide) stimulated by the target OVCAR3 (FRα⁺MSLN⁺) or non-target A2780cis (FRα^{neg}MSLN^{neg}) cells was fit using a four-parameter logistic model, Luc2=Luc2ₘᵢₙ+{Luc2ₘₐₓ−Luc2ₘᵢₙ}/{1+10\hat{ }[b*(log₁₀[η(E:T)₅₀]−X)]}.

Various embodiments are implemented in accordance with the underlying Provisional Application Ser. No. 63/106,838, entitled “Modular Antigen-Specific NK-Cell Biofactory for In Situ Synthesis of Engineered Proteins,” filed Oct. 28, 2020, to which benefit is claimed and which is fully incorporated herein by reference for its general and specific teachings. For instance, embodiments herein and/or in the Provisional Application can be combined in varying degrees (including wholly). Reference can also be made to the experimental teachings and underlying references provided in the underlying Provisional Application. Embodiments discussed in the Provisional Application are not intended, in any way, to be limiting to the overall technical disclosure, or to any part of the claimed disclosure unless specifically noted.

Although specific examples have been illustrated and described herein, a variety of alternate and/or equivalent implementations can be substituted for the specific examples shown and described without departing from the scope of the present disclosure. This application is intended to cover any adaptations or variations of the specific examples discussed herein.

1. A genetically engineered natural killer (NK) cell comprising an exogenous polynucleotide sequence that includes, in operative association:
a receptor element that encodes a chimeric antigen receptor (CAR) comprising an extracellular antigen binding domain operably linked to a transmembrane domain, and an intracellular signaling domain, wherein the extracellular antigen binding domain recognizes a surface antigen on a surface of a target cell;
an actuator element that encodes a transcription factor binding site that upregulates synthesis of an effector protein in response to the antigen binding domain of the CAR binding to the antigen of the target cell; and
an effector element that encodes the effector protein operably linked to a signal peptide, wherein, in response to the antigen binding domain of the CAR binding to the antigen of the target cell, the engineered NK cell is configured to activate and, to synthesize and secrete the effector protein.

2. The genetically engineered NK cell of claim 1, wherein the genetically engineered NK cell is configured to synthesize and secrete an amount of the effector protein as a function of an amount of the target cell present.

3. The genetically engineered NK cell of claim 2, wherein the amount of the effector protein is proportional to the amount of target cell present in situ.

4. The genetically engineered NK cell of claim 1, wherein the signal peptide is upstream of the effector protein and is non-native to the effector protein.

5. The genetically engineered NK cell of claim 1, wherein the signal peptide is native to the effector protein.

6. The genetically engineered NK cell of claim 1, wherein the intracellular signaling domain, the actuator element, and the signal peptide are constant domains and the extracellular antigen binding domain and the effector protein are variable domains.

7. The genetically engineered NK cell of claim 1, wherein the actuator element is bound to the effector element and the NK cell includes a NK-92MI cell.

8. The genetically engineered NK cell of claim 1, wherein the exogenous polynucleotide sequence includes the actuator element bound to the effector element bound to the receptor element.

9. The genetically engineered NK cell of claim 1, wherein the effector protein is selected from a detectable reporter protein, a therapeutic protein, a downstream signaling protein, and a combination thereof.

10. The genetically engineered NK cell of claim 1, wherein the intracellular signaling domain includes one or more of an intracellular signaling portion of a CD28, an intracellular signaling portion of a 4-1BB and an intracellular signaling portion of a CD3 zeta.

11. The genetically engineered NK cell of claim 1, wherein the transcription factor binding site is selected from the group consisting of:
a nuclear factor of activated T-cell (NFAT) response element, a serum response element (SRE), and a cyclic AMP response element (CRE).

12. A population of genetically engineered natural killer (NK) cells, each of the genetically engineered NK cells of the population comprising an exogenous polynucleotide sequence that includes an actuator element bound to an effector element bound to a receptor element, wherein:
the receptor element encodes a chimeric antigen receptor (CAR) comprising an extracellular antigen binding domain operably linked to a transmembrane domain, and an intracellular signaling domain, wherein the extracellular antigen binding domain recognizes a surface antigen on a surface of a target cell;
the actuator element encodes a transcription factor binding site that upregulates synthesis of an effector protein in response to the antigen binding domain of the CAR binding to the antigen of the target cell; and
the effector element encodes the effector protein operably linked to a signal peptide, wherein, in response to the antigen binding domain of the CAR binding to the antigen of the target cell, the population of engineered NK cells are configured to activate and, in response, to synthesize and secrete a calibrated amount of the effector protein based on a presence of the target cell.

13. The population of genetically engineered NK cells of claim 12, wherein the exogenous polynucleotide sequence includes the actuator element bound to and upstream from the effector element, and the effector element bound to and upstream from the receptor element, and wherein the signal peptide is upstream from the effector protein.

14. The population of genetically engineered NK cells of claim 12, wherein the effector protein is a therapeutic protein that acts directly upon the target cell, the therapeutic protein being selected from the group consisting of:
a cytotoxic protein, an immunostimulatory protein, and an immunosuppressive protein.

15. The population of genetically engineered NK cells of claim 12, wherein the calibrated amount of the effector protein is a function of an amount of the target cell present in a plurality of cells or in a sample.

16. A method comprising
contacting a plurality of cells with a volume of a genetically engineered natural killer (NK) cell, wherein the genetically engineered NK cell comprises a polynucleotide sequence that includes:
a receptor element that encodes a chimeric antigen receptor (CAR) comprising an extracellular antigen binding domain operably linked to a transmembrane domain, and an intracellular signaling domain, wherein the extracellular antigen binding domain recognizes a surface antigen on a surface of a target cell from the plurality of cells;
an actuator element that encodes a transcription factor binding site; and
an effector element that encodes an effector protein operably linked to a signal peptide;
in response to contacting the plurality of cells with the genetically engineered NK cell and a presence of the target cell within the plurality of cells, causing binding of the receptor element to an antigen on a surface of the target cell; and
in response to the antigen binding domain of the CAR binding to the antigen of the target cell,
initiating expression of the effector element by the actuator element to synthesize the effector protein and the secretor peptide; and
secreting the effector protein by the secretor peptide.

17. The method of claim 16, further including detecting expression of the effector protein, wherein detectable expression of the effector protein indicates the presence of the target cell.

18. The method of claim 16, further including, in response to the antigen binding domain of the CAR binding to the antigen of the target cell, activating the NK cell and, in response, synthesizing and secreting a calibrated amount of the effector protein based on the presence of the target cell.

19. The method of claim 18, wherein the amount of the effector protein is proportional to an amount of the target cell present within the plurality of cells.

20. The method of claim 16, wherein the effector protein includes a therapeutic protein that acts directly on the target cell, and the method further includes neutralizing the target cell by the therapeutic protein.
